(19) United States
(12) Patent Application Publication
(10) Pub. No.: US 2019/0352429 A1
(43) Pub. Date: Nov. 21, 2019

(54) PREFERRED PAIRING OF ANTIBODY DOMAINS

(71) Applicant: MERCK PATENT GMBH, Darmstadt (DE)

(72) Inventors: Florian RÜKER, Vienna (AT); Maximilian BÖNISCH, Vienna (AT)

(73) Assignee: MERCK PATENT GMBH, Darmstadt (DE)

(21) Appl. No.: 16/476,380

(22) PCT Filed: Feb. 2, 2018
(86) PCT No.: PCT/EP2018/052624

§ 371 Date: Jul. 8, 2019

(30) Foreign Application Priority Data
Feb. 2, 2017 (EP) 17154388.7

(51) Int. Cl. C07K 16/46; C07K 16/28

(52) U.S. Cl. CPC C07K 16/468; C07K 16/2863; C07K 16/2809; C07K 2317/94; C07K 2317/31; C07K 2317/51; C07K 2317/515; C07K 2317/21

(57) ABSTRACT

An antigen-binding molecule (ABM) comprising a cognate LC/HC dimer of an antibody light chain (LC) composed of a VL and a CL antibody domain, associated to an antibody heavy chain (HC) comprising at least a VH and a CH1 antibody domain, which association is through pairing the VL and VH domains and the CL and CH domains, wherein the amino acids at the position 18 in the CL domain and at the position 26 in the CH1 domain are of opposite polarity, wherein numbering is according to the IMGT.

The invention relates to an antigen-binding molecule (ABM) which comprises human antibody domain sequences, in particular comprising a cognate dimer of an antibody light chain composed of a VL and a CL antibody domain, associated to an antibody heavy chain comprising at least a VH and a CH1 antibody domain, which association is through pairing the VL and VH domains and the CL and CH1 domains, wherein a preferred pairing is supported by certain point mutations in the CL and CH1 domains.

### BACKGROUND

Monoclonal antibodies have been widely used as therapeutic antigen-binding molecules. The basic antibody structure will be explained here using as example an intact IgG1 immunoglobulin.

Two identical heavy (H) and two identical light (L) chains combine to form the Y-shaped antibody molecule. The heavy chains each have four domains. The amino terminal variable domains (VH) are at the tips of the Y. These are followed by three constant domains: CH1, CH2, and the carboxy-terminal CH3, at the base of the Y's stem. A short stretch, the switch, connects the heavy chain variable and constant regions. The hinge connects CH2 and CH3 (the Fc fragment) to the remainder of the antibody (the Fab fragments). One Fc and two identical Fab fragments can be produced by proteolytic cleavage of the hinge in an intact antibody molecule. The light chains are constructed of two domains, variable (VL) and constant (CL), separated by a switch.

Disulfide bonds in the hinge region connect the two heavy chains. The light chains are coupled to the heavy chains by additional disulfide bonds. Asn-linked carbohydrate moieties are attached at different positions in constant domains depending on the class of immunoglobulin. For IgG1 two disulfide bonds in the hinge region, between Cys235 and Cys238 pairs, unite the two heavy chains. The light chains are coupled to the heavy chains by two additional disulfide bonds, between Cys220 (EU Index numbering) or Cys233 (numbering according to Kabat) in the CH1 domains and Cys214 in the CL domains (EU index and Kabat numbering). Carbohydrate moieties are attached to Asn306 of each CH2, generating a pronounced bulge in the stem of the Y.

These features have profound functional consequences. The variable regions of both the heavy and light chains (VH) and (VL) lay at the N-terminal region, i.e. the “tips” of the Y, where they are positioned to react with antigen. This tip of the molecule is the side on which the N-terminus of the amino acid sequence is located. The stem of the Y projects in a way to efficiently mediate effector functions such as the activation of complement and interaction with Fc receptors, or ADCC and ADCP. Its CH2 and CH3 domains bulge to facilitate interaction with effector proteins. The C-terminus of the amino acid sequence is located on the opposite side of the tip, which can be termed “bottom” of the Y.

Two types of light chain, termed lambda (λ) and kappa (κ), are found in antibodies. A given immunoglobulin either has κ chains or λ chains, never one of each. No functional difference has been found between antibodies having λ or κ light chains.

Each domain in an antibody molecule has a similar structure of two beta sheets packed tightly against each other in a compressed antiparallel beta barrel. This conserved structure is termed the immunoglobulin fold. The immunoglobulin fold of constant domains contains a 3-stranded sheet packed against a 4-stranded sheet. The fold is stabilized by hydrogen bonding between the beta strands of each sheet, by hydrophobic bonding between residues of opposite sheets in the interior, and by a disulfide bond between the sheets. The 3-stranded sheet comprises strands C, F, and G, and the 4-stranded sheet has strands A, B, E, and D. The letters A through G denote the sequential positions of the beta strands along the amino acid sequence of the immunoglobulin fold.

The fold of variable domains has 9 beta strands arranged in two sheets of 4 and 5 strands. The 5-stranded sheet is structurally homologous to the 3-stranded sheet of constant domains, but contains the extra strands C′ and C″. The remainder of the strands (A, B, C, D, E, F, G) have the same topology and similar structure as their counterparts in constant domain immunoglobulin folds. A disulfide bond links strands B and F in opposite sheets, as in constant domains.

The variable domains of both light and heavy immunoglobulin chains contain three hypervariable loops, or complementarity-determining regions (CDRs). The three CDRs of a V domain (CDR1, CDR2, CDR3) cluster at one end of the beta barrel. The CDRs are loops that connect beta strands B-C, C′-C″, and F-G of the immunoglobulin fold. The residues in the CDRs vary from one immunoglobulin molecule to the next, imparting antigen specificity to each antibody.

The VL and VH domains at the tips of antibody molecules are closely packed such that the 6 CDRs (3 on each domain) cooperate in constructing a surface (or cavity) for antigen-specific binding. The natural antigen binding site of an antibody thus is composed of the loops which connect strands B-C, C′—C″, and F-G of the light chain variable domain and strands B-C, C′-C″, and F-G of the heavy chain variable domain.

The loops which are not CDR-loops in a native immunoglobulin, or not part of the antigen-binding pocket as determined by the CDR loops and optionally adjacent loops within the CDR loop region, do not have antigen binding or epitope binding specificity, but contribute to the correct folding of the entire immunoglobulin molecule and/or its effector or other functions and are therefore called structural loops.

Prior art documents show that the immunoglobulin-like scaffold has been employed so far for the purpose of manipulating the existing antigen binding site, thereby introducing novel binding properties. In most cases the CDR regions have been engineered for antigen binding, in other words, in the case of the immunoglobulin fold, only the natural antigen binding site has been modified in order to change its binding affinity or specificity. A vast body of literature exists which describes different formats of such manipulated immunoglobulins, frequently expressed in the form of single-chain Fv fragments (scFv) or Fab fragments, either displayed on the surface of phage particles or solubly expressed in various prokaryotic or eukaryotic expression systems.

Antibody constructs are currently in development for improved therapeutics recognizing two different targets.

Davis et al (Protein Engineering, Design & Selection 2010, 23(4) 195-202) describe a heterodimeric Fc platform that supports the design of bispecific and asymmetric fusion proteins by using strand-exchange engineered domain (SEED) CH3 heterodiomers. These derivatives of human IgG and IgA CH3 domains create complementary human SEED CH3 heterodimers that are composed of alternating segments of human IgA and IgG sequences. The SEED engineering is further described in WO2007/110205A2 and EP1999154B1. WO2010/136172A1 discloses tri- or tetra specific antibodies that comprise one or two single-chain Fab connected to the C-terminus of the Fc part of the antibody.

Beck et al, (Nature Reviews Immunology, vol. 10, no. 5, 1 May 2010, pp 345-352) describes next generation therapeutic antibodies, and particularly refers to different types of bispecific antibodies.

Ridgway et al. (Protein Engineering, vol. 9, no. 7, 1996, pp 617-621) describes “knobs into-holes” engineering of antibody CH3 domains for heavy chain heterodimerization.

Von Kreudenstein et al. (Landes Bioscience, vol. 5, no. 5, 2013, pp 646-654) describe a bispecific antibody scaffold based on a heterodimeric Fc engineered for stability.

Liu et al. (Journal Of Biological Chemistry 2015, 290:7535-7562) describe a strategy of making monovalent bispecific heterodimeric IgG antibodies by electrostatic mechanism. Heterodimeric IgG molecules derived from anti-HER2 and anti-EGFR antibodies with correct light chain (LC) and heavy chain (HC) pairings were produced by transiently and stably transfected mammalian cells. Specific pairing of LC and HC was driven by switching polar or hydrophobic residues at the VH-VL and CH1-CL interfaces. Each of the engineered variants was characterized by a series of point mutations, among them in the VH and VL domains. In addition, point mutations were engineered in the CH1 domain e.g., K147D, and in the CL (Ckappa or CK) domain e.g., T180K, (numbering according to the EU index). Some variants contained inter alia point mutations at positions 147 in the CH1 domain and 180 or 131 in the Ckappa domain.

WO2014/081955 further discloses such heterodimeric antibodies comprising one or more substitutions in each of the following domains: a first and second CH3 domain, a CH1 domain, a CL domain, a VH domain and a VL domain.

Lewis et al. (Nature Biotechnology 2014, 32: 191-198) describe the generation of bispecific IgG antibodies by structure-based design of an orthogonal Fab interface. Bispecific IgG with improved HC-LC pairing were produced. It was found that the variable domains dominated the specific assembly of heavy chains and light chains. Two distinct designs were employing point mutations in each of the VH, VL, CH1 and CL domains. One of the designs contained inter alia point mutations at positions 146 in the CH1 domain and 129 in the Clambda domain (numbering according to Kabat).

Dillon et al. (MAbs 2016; DOI:10.1080/19420862.2016.1267089) describe the production of bispecific IgG of different isotypes and species of origin in single mammalian cells, and designs which facilitate selective Fab arm assembly in conjunction with previously described knobs-into-holes mutations for preferential heavy chain heterodimerization.

Bispecific antibodies of the above described designs necessarily combine a series of point mutations to stabilize the IgG structure, including the predominant point mutations positioned in the VH and VL domains. It would be desirable to engineer bispecific antibodies wherein correct pairing is already supported by point mutations in the CH1 and CL domains only.

### SUMMARY OF THE INVENTION

It is the objective of the present invention to provide improved pairing of an antibody heavy and light chain, which supports the correct pairing of HC and LC, while leaving the framework of the VH and VL domains unchanged. Such improved pairing would facilitate the production of bispecific antibodies.

The object is solved by the subject of the present invention.

According to the invention, there is provided an antigen-binding molecule (ABM) comprising a cognate LC/HC dimer of an antibody light chain (LC) composed of a VL and a CL antibody domain, associated to an antibody heavy chain (HC) comprising at least a VH and a CH1 antibody domain, which association is through pairing the VL and VH domains and the CL and CH1 domains, wherein the amino acids at the position 18 in the CL domain and at the position 26 in the CH1 domain are of opposite polarity, wherein numbering is according to the IMGT.

Specifically, the cognate LC/HC dimer is characterized by cognate domains, which are paired to form a cognate (domain) pair. It is specifically understood that the LC/HC dimer is cognate, because the monomeric CL and CH1 domains are cognate or matching counterparts, preferably recognizing each other to produce a pair of CL and CH1 domains as compared to wild-type domains. Specifically, the CL domain as described herein is preferably pairing with the cognate CH1 domain; and the CH1 domain as described herein is preferably pairing with the cognate CL domain.

According to a specific aspect, the ABM is characterized by cognate CL and CH1 antibody domains that preferably pair with each other through attractive forces, and do not preferably pair with other counterpart domains which are discognate or wild-type because of repulsive forces. Thereby, false pairing of counterpart antibody domains that are wild-type antibody domains, or which have been rendered non-cognate (repulsive to decrease the likelihood of assembly) through respective point mutations is greatly reduced.

Specifically, the cognate CL and CH1 domains are characterized by the opposite polarity at the recited amino acid positions, in particular such that

a) the amino acid residue at the position 18 in the CL domain is of positive polarity, in particular any of R, H, or K; and the amino acid residue at the position 26 in the CH1 domain is of negative polarity, in particular any of D or E; or

b) the amino acid residue at the position 18 in the CL domain is of negative polarity, in particular any of D or E; and the amino acid residue at the position 26 in the CH1 domain is of positive polarity, in particular any of R, H, or K.

Specifically, the ABM comprises one or two point mutations, which are any of or both of the point mutations at position 18 in the CL domain and the point mutations at position 26 in the CH1 domain.

Unless indicated otherwise, the positions are herein numbered according to the IMGT system (Lefranc et al., 1999, Nucleic Acids Res. 27: 209-212). Numbering of the positions indicated in the claims corresponds to numbering according to Kabat and the EU index of Kabat as indicated in the following table. An explanation of the Kabat numbering scheme can be found in Kabat, E A, et aL, Sequences of proteins of immunological interest (NIH publication no. 91-3242, 5^{th} edition (1991)).

| CH1 | | | CL | | |
|---|---|---|---|---|---|
| IMGT | Kabat | EU | IMGT | Kabat | EU |
| 26 | 145 | 147 | 18 | 129 | 129 |

The indicated positions surprisingly turned out to be dominant when constructing a Fab arm wherein the HC and LC assemble (pair) with improved affinity. Prior art constructs involved different pairs of CH1 and CL point mutations located on different positions, which were engineered besides dominant VH and VL point mutations. By establishing opposite polarities at the indicated CL and CH1 positions, a cognate pair of mutated CL and CH1 domains (herein understood as cognate domains or cognate pair) is preferably produced. At the same time, false cognate pairing or pairing with the wild-type CL and CH1 domains is markedly reduced.

Specifically, the ABM is characterized as follows:

A

a) the CL domain is Ckappa comprising an amino acid sequence with at least 90% sequence identity to SEQ ID 1 which contains at least the point mutation T18X, wherein X is any of R, H, or K; and

b) the CH1 domain comprises an amino acid sequence with at least 90% sequence identity to SEQ ID 3 which contains at least the point mutation K26X, wherein X is any of D, or E;

or B

a) the CL domain is Clambda comprising an amino acid sequence with at least 90% sequence identity to SEQ ID 2 which contains at least the point mutation K18X, wherein X is any of D, or E; and

b) the CH1 domain comprises an amino acid sequence with at least 90% sequence identity to SEQ ID 3 wherein K at position 26 is not substituted by any other amino acid, or which contains at least the point mutation K26X, wherein X is any of R, or H;

or C

a) the CL domain is Clambda comprising an amino acid sequence with at least 90% sequence identity to SEQ ID 2 wherein K at position 18 is not substituted by any other amino acid, or which contains at least the point mutation K18X, wherein X is any of R, or H; and

b) the CH1 domain comprises an amino acid sequence with at least 90% sequence identity to SEQ ID 3 which contains at least the point mutation K26X,

wherein X is any of D, or E;

wherein numbering is according to the IMGT.

Specifically, the CL and CH1 domains are of human origin, specifically of a human IgG or IgG1 molecule, in particular functionally active variants characterized by at least 90% sequence identity to the naturally-occurring human sequence and one or more point mutations, such as described herein, and specifically further characterized by the beta-barrel structure of the antibody domain which resembles the structure of respective domains in the human IgG, IgM or IgE structure, in particular a human IgG1 structure.

Functionally active variants of any of the Ckappa, Clambda, or CH1 domains as described herein are specifically characterized by the antibody domain structure capable of pairing with the corresponding matching antibody domain, in particular wherein

A

a) the CL domain variant is a Ckappa variant comprising an amino acid sequence with at least 90% sequence identity to SEQ ID 1 and which contains the point mutation T18X, wherein X is any of R, H, or K; is capable of pairing with

b) a CH1 domain consisting of an amino acid sequence identified as SEQ ID 3, except for a point mutation K26X, wherein X is any of D, or E;

or B

a) the CL domain variant is a Clambda variant comprising an amino acid sequence with at least 90% sequence identity to SEQ ID 2 and which contains the point mutation K18X, wherein X is any of D, or E; is capable of pairing with

b) a CH1 domain consisting of an amino acid sequence identified as any of SEQ ID 3, or SEQ ID 3 except for the point mutation K26X, wherein X is any of R, or H;

or C

a) the CL domain variant is a Clambda variant comprising an amino acid sequence with at least 90% sequence identity to SEQ ID 2 wherein K at position 18 is not substituted by any other amino acid, or which contains the point mutation K18X, wherein X is any of R, or H; is capable of pairing with

b) a CH1 domain consisting of an amino acid sequence identified as SEQ ID 3, except for the point mutation K26X, wherein X is any of D, or E.

Specifically, the Ckappa amino acid sequence (herein also referred to as wild-type or parent) is identified by SEQ ID 1.

Specifically, the Clambda amino acid sequence (herein also referred to as wild-type or parent) is identified by SEQ ID 2.

Specifically, the CH1 amino acid sequence (herein also referred to as wild-type or parent) is identified by SEQ ID 3.

Specifically, the CL domain is characterized by the CL sequence of human IgG1 or an engineered functionally active variant thereof comprising one or more point mutations, preferably up to 10 point mutations, in particular any of 1, 2, 3, 4, 5, 6, 7, 8, 9, or 10 point mutations.

Specifically, the CH1 domain is characterized by the CH1 sequence of human IgG1 or an engineered functionally active variant thereof comprising one or more point mutations, preferably up to 10 point mutations, in particular any of 1, 2, 3, 4, 5, 6, 7, 8, 9, or 10 point mutations.

Specifically, any or each of the CL and CH1 domains is characterized by the respective human IgG1 sequence or an engineered functionally active variant thereof comprising one or more point mutations, preferably up to 10 point mutations, in particular any of 1, 2, 3, 4, 5, 6, 7, 8, 9, or 10 point mutations.

Specifically, the dimer comprises at least one interdomain disulfide bridge between the CL and CH1 domains. Specifically, the interdomain disulphide bridges are bridging Cys220 (EU Index numbering) or Cys233 (numbering according to Kabat) in the CH1 domains and Cys214s in the CL domains. (EU index and Kabat numbering).

Specifically, the CL domain further comprises the point mutation F7X, wherein X is any of S, A, or V, and which CH1 domain further comprises the point mutation A20L, wherein numbering is according to the IMGT. Such further point mutations additionally support the preferred pairing of the cognate CL and CH1 domains.

Specifically, the VL and VH domains in the ABM do not contain any point mutation changing the polarity of an amino acid in the interface region that provide for the interdomain contact when pairing the VL and VH domains, thereby forming the antigen-binding site.

Specifically, the ABM comprises a functional antigen-binding site composed of a VH/VL domain pair, capable of binding a target with a high affinity and a KD of less than any of 10⁻⁶M, 10⁻⁷M, 10⁻⁸M, 10⁻⁹M, or 10⁻¹⁰M. Specifically, the ABM is a bispecific or heterodimeric antibody targeting two different antigens, wherein each of the antigens is recognized by the antibody with a KD of less than any of 10⁻⁶M, 10⁻⁷M, 10⁻⁸M, 10⁻⁹M, or 10⁻¹⁰M.

Specifically, the HC further comprises at least one CH2 and at least one CH3 domain. In particular, the HC is extended by the CH2 domain and further extended by the CH3 domain, namely a sequence of CH2-CH3 domains is further paired with another antibody chain comprising a CH2-CH3 domain, such as to form a Fc region consisting of a dimer of CH2-CH3 domains and respective chains. Specifically, the HC is extended by fusing a CH2 domain to the C-terminus of the CH1 domain with or without using a linker or hinge region. Specifically, the HC is further extended by fusing a CH3 domain to the C-terminus of the CH2 domain with or without using a linker. In some cases, the HC is further extended by fusing a CH4 domain to the C-terminus of the CH3 domain, with or without using a linker.

Specifically, the ABM comprises a hinge region, preferably a human hinge region e.g. a human IgG1 hinge region, such as comprising or consisting of the amino acid sequence identified as SEQ ID 4.

The linkage of domains is specifically by recombinant fusion or chemical linkage. Specific linkage may be through linking the C-terminus of one domain to the N-terminus of another domain, e.g. wherein one or more amino acid residues in the terminal regions are deleted to shorten the domain size, or extended to increase flexibility of the domains.

Specifically, the shortened domain sequence comprises a deletion of the C-terminal and/or N-terminal region, such as to delete at least 1, 2, 3, 4, or 5, up to 6, 7, 8, 9, or 10 amino acids.

Specifically, a linking sequence, which is a linker or a hinge region or at least part of the hinge region of an immunoglobulin may be used, such as a peptidic linker e.g. including at least 1, 2, 3, 4, or 5 amino acids, up to 10, 15, or 20 amino acids. A linking sequence is herein also referred to as “junction”. A domain may be extended by a linker e.g. through an amino acid sequence that originates from the N-, or C-terminal region of an immunoglobulin domain that would natively be positioned adjacent to the domain, such as to include the native junction between the domains. Alternatively, the linker may contain an amino acid sequence originating from the hinge region. However, the linker may as well be an artificial sequence, e.g. consisting of serial Gly and/or Ser amino acids, preferably with a length of 5 to 20 amino acids, preferably 8 to 15 amino acids.

According to a specific aspect, the ABM is an antibody or immunoglobulin comprising the structure of a naturally-occurring immunoglobulin or an immunoglobulin-like scaffold, which ABM is characterized by at least one (preferably two) antigen-binding site(s) and a structure composed of antibody domains interlinked to heavy and light chains with or without suitable linking sequences, wherein a HC dimerizes to a LC to form the at least one antigen-binding site, and optionally wherein two HCs dimerize to form an Fc region.

Specifically, the ABM is any of an antibody Fab or (Fab)₂ fragment, or a full-length antibody comprising an Fc part or Fc region, preferably wherein the ABM is a full-length IgG, IgM, or IgE antibody, in particular any of IgG1, IgG2, IgG3, or IgG4. Specifically, the ABM comprises one or two Fab arms or Fab fragments (Fab parts) in any suitable order. According to specific embodiments, the ABM may even comprise more than two Fab arms, e.g. three or four Fab arms, wherein at least one or only one of the Fab arms comprises the cognate LC/HC pair and the cognate CL and CH1 domains as described herein. According to a specific embodiment, the other Fab arm comprises wild-type (naturally-occurring) CL and CH1 domains.

According to a specific embodiment, the ABM comprises only one cognate LC/HC dimer, wherein the HC is further dimerized with an Fc chain comprising CH2-CH3, optionally further comprising CH4, thereby obtaining the Fc region. Such ABM is specifically a monovalent, monospecific antibody characterized by only one Fab arm and the Fc region.

According to another specific embodiment, the ABM comprises at least two LC/HC dimers, wherein only one of the LC/HC dimers is characterized by the cognate LC/HC dimer and the cognate CL and CH1 domains (i.e. the cognate CL/CH1 pair) as described herein. Alternatively, the ABM is composed of a first LC/HC dimer comprising a first cognate LC/HC dimer comprising a first cognate CL/CH1 pair characterized by the point mutations described herein, and a second cognate LC/HC dimer comprising a second cognate CL/CH1 pair characterized by point mutations described herein, which are different from those of the first cognate CL/CH1 pair, such that the first cognate CL and CH1 domains preferably pair with each other, and the second cognate CL and CH1 domains preferably pair with each other, however, the CL domain of the first cognate CL/CH1 pair does not preferably pair with (or even is repulsive to) the CH1 domain of the second cognate CL/CH1 pair, and the CH1 domain of the first cognate CL/CH1 pair does not preferably pair with (or even is repulsive to) the CL domain of the second cognate CL/CH1 pair.

According to a specific embodiment, the ABM comprises two different Fab arms, thereby providing for two different Fv structures, each with specific binding characteristics. Specifically, the ABM is a heterodimeric or bispecific antibody targeting two different antigens or two different epitopes of an antigen.

The invention further provides for a heterodimeric or bispecific antibody comprising a first and a second Fab arm recognizing different antigens or epitopes, wherein only one of the first and second Fab arms comprises the cognate LC/HC dimer of the ABM as described herein. Specifically, the heterodimeric antibody is a bispecific antibody or immunoglobulin, or an antigen-binding fragment thereof, such as a bispecific full-length immunoglobulin, or a (Fab)₂.

Specifically, the ABM is a bispecific antibody, wherein the first target is any of CD3, CD16 or Her2neu, and the second target is EGFR.

A Fab arm is herein particularly understood as a dimer of a HC consisting of a VH-CH1 domain sequence and a LC consisting of a VL-CL (kappa or lambda) domain sequence, with or without any disulfide bridges, a hinge domain and/or linker sequences connecting antibody domains. A Fab arm is typically understood as a Fab fragment (or Fab part) when cleaved from an antibody. The Fab arm is specifically characterized by only one antigen-binding site formed by pairing the VH and VL domains, which is capable of binding the target only monospecifically and monovalently.

Specifically, only one of the first and second Fab arms in the bispecific antibody comprises

a) the point mutation F7X in the CL domain, wherein X is any of S, A, or V; and

b) the point mutation A20L in the CH1 domain;

wherein numbering is according to the IMGT.

Such point mutations at the positions 7 and 20 indicated above are herein understood as supportive point mutations, which actually do not change the polarity of the amino acid residue, but the sterical characteristics matching the counterpart amino acid residue dimension.

Specifically, the CL domain comprising the supportive F7X point mutation indicated above, wherein X is any of S, A, or V, attracts and preferably pairs with the counterpart CH1 domain comprising the supportive A20L point mutation, but unpreferably pairs with a wild-type CH1 domain, or a CH1 domain that does not contain the A20L point mutation.

Specifically, the CH1 domain comprising the supportive A20L point mutation indicated above, attracts and preferably pairs with the counterpart CL domain comprising the supportive F7X point mutation indicated above, wherein X is any of S, A, or V, but unpreferably pairs with a wild-type CL domain, or a CL domain that does not contain the F7X point mutation indicated above, wherein X is any of S, A, or V.

Specifically, the heterodimeric antibody is characterized by

A

a) said first Fab arm comprises the cognate LC/HC dimer described herein which is specifically characterized by the point mutations identified above, in particular one or two point mutations providing for the amino acid residue at the position 18 in the CL domain and the amino acid residue at the position 26 in the CH1 domain which are of opposite polarity, wherein the CL and CH1 domains further comprise the supportive point mutations identified above, in particular the point mutation F7X in the CL domain, wherein X is any of S, A, or V; and the point mutation A20L in the CH1 domain; and

b) said second Fab arm does not comprise any of the point mutations of a), or

B

a) said first Fab arm comprises the cognate LC/HC dimer described herein which is specifically characterized by the point mutations identified above, in particular one or two point mutations providing for the amino acid residue at the position 18 in the CL domain and the amino acid residue at the position 26 in the CH1 domain which are of opposite polarity, wherein the CL and CH1 domains do not further comprise the supportive point mutations identified above, in particular the point mutation F7X in the CL domain, wherein X is any of S, A, or V; and the point mutation A20L in the CH1 domain; and

b) said second Fab arm comprises the supportive point mutations identified above, in particular the point mutation F7X in the CL domain, wherein X is any of S, A, or V; and the point mutation A20L in the CH1 domain.

Such bispecific construct of A is specifically characterized by the point mutations described herein for preferred pairing of cognate CL and CH1 domains of the cognate LC/HC dimer, which are engineered in only one of the two Fab arms (i.e. the first Fab arm), thereby unfavourably pairing or attaching to any of the HC or LC of the other Fab arm (i.e. the second Fab arm).

Such bispecific construct of B is specifically characterized by a first Fab arm which comprises the cognate LC/HC dimer as described herein characterized by one or two point mutations at the position 18 in the CL domain and at the position 26 in the CH1 domain thereby obtaining amino acid residues at these positions which are of opposite polarity, and a second Fab arm which comprises the supportive point mutations, thereby

a) the HC of the first Fab arm favourably pairs with or attaches to the LC of the first Fab arm, and unfavourably pairs with or attaches to the LC of the second Fab arm; and

b) the LC of the first Fab arm favourably pairs with or attaches to the HC of the first Fab arm, and unfavourably pairs with or attaches to the HC of the second Fab arm;

and vice versa, meaning that

c) the HC of the second Fab arm favourably pairs with or attaches to the LC of the second Fab arm, and unfavourably pairs with or attaches to the LC of the first Fab arm; and

d) the LC of the second Fab arm favourably pairs with or attaches to the HC of the second Fab arm, and unfavourably pairs with or attaches to the HC of the first Fab arm.

According to a specific embodiment, both, the first and second Fab arms comprise one or two point mutations at the position 18 in the CL domain and at the position 26 in the CH1 domain thereby obtaining amino acid residues at these positions which are of opposite polarity, yet wherein the point mutations in the first and second Fab arms are different, thereby producing

a) a first Fab arm which comprises a CL domain, wherein the amino acid residue at position 18 is of positive polarity specifically recognizing the CH1 domain wherein the amino acid residue at position 26 is of negative polarity; and

b) a second Fab arm which comprises a CL domain, wherein the amino acid residue at position 18 is of negative polarity specifically recognizing the CH1 domain wherein the amino acid residue at position 26 is of positive polarity;

optionally, wherein the supportive point mutations are either in the first Fab arm or in the second Fab arm.

Further embodiments refer to bispecific constructs, wherein

a) a first Fab arm which comprises a CL domain, wherein the amino acid residue at position 18 is of positive polarity specifically recognizing the CH1 domain wherein the amino acid residue at position 26 is of negative polarity; and

b) a second Fab arm wherein the amino acid residue at position 18 in the CL domain and/or the amino acid residue at position 26 in the CH1 domain is of no charge, in particular any of N, C, Q, G, S, T, W, or Y; or non-polar, in particular any of A, I, L, M, F, P or V;

optionally, wherein the supportive point mutations are either in the first Fab arm or in the second Fab arm.

According to a specific aspect, the ABM described herein, in particular the heterodimeric antibody described herein, comprises two HCs each comprising a CH2 and a CH3 domain, and optionally a CH4 domain, which HCs dimerize into an Fc region.

The Fc region is specifically characterized by a dimer of Fc chains each characterized by comprising the chain of CH2-CH3 antibody domains, which dimer can be a homodimer or a heterodimer, e.g. wherein a first Fc chain differs from a second Fc chain in at least one point mutation in the CH2 and/or CH3 domains.

Specifically, the Fc region comprises two CH3 domains which are engineered to introduce and/or are characterized by one or more of the following:

a) strand-exchange engineered domain (SEED) CH3 heterodimers that are composed of alternating segments of human IgA and IgG CH3 sequences;

b) one or more knob or hole mutations, preferably any of T366Y/Y407′T, F405A/T394′W, T366Y:F405A/T394′W:Y407′T, T366W/Y407′A and S354C:T366W/Y349′C:T366′S:L368′A:Y407V;

c) a cysteine residue in the first CH3 domain that is covalently linked to a cysteine residue in the second CH3 domain, thereby introducing an interdomain disulfide bridge, preferably linking the C-terminus of both CH3 domains;

d) one or more mutations where repulsive charge suppresses heterodimer formation, preferably any of: K409D/D399′K, K409D/D399′R, K409E/D399′K, K409E/D399′R, K409D:K392D/D399′K:E356′K or K409D:K392D:K370D/D399′K:E356′K:E357′K; and/or

e) one or more mutations selected for heterodimer formation and/or thermostability, preferably any of:

T350V:L351Y:F405A:Y407V/T350V:T366L:K392L:T394W,

T350V:L351Y:F405A:Y407V/T350V:T366L:K392M:T394W,

L351Y:F405A:Y407V/T366L:K392M:T394W,

F405A:Y407V/T366L:K392M:T394W, or

F405A:Y407V/T366L:T394W,

wherein numbering is according to the EU index of Kabat.

Such CH3 mutations are engineered to produce two different Fc chains and HCs (differing at least by a different sequence of the CH3 domains), respectively, which preferably pair with each other, thereby obtaining a heterodimer of the Fc chains or HCs, substantially reducing the tendency of producing a HC homodimer, i.e. a dimer of two HCs of the same sequence.

In the specification of the CH3 point mutations described herein, the “slash” differentiates the point mutations on one chain or one domain from the point mutations from the other chain or other domain of the respective pair; the “indent” in the amino acid position numbering signifies the second chain or dimer of the heterodimer. The “colon” identifies the combination of point mutations on one of the chains or domains, respectively.

Any of the mutations selected for heterodimer formation and/or thermostability as mentioned above or further mutations in accordance with the disclosure of Von Kreudenstein et al. (Landes Bioscience, vol. 5, no. 5, 2013, pp 646-654) can be used.

Preferably, either (i) a knob; or (ii) a hole mutation, or (iii) a knob and hole mutation, is engineered on one chain or domain, and the counterpart (i) hole, or (ii) knob mutation, or (iii) hole and knob mutation, is engineered on the other chain of the heterodimer.

Specifically, a pair of CH3 domains comprising one or two engineered CH3 domains may comprise more than one (additional) interdomain disulfide bridges, e.g. 2, or 3, connecting the pair of two CH3 domains.

Specifically, different mutations (according to a) above) are engineered in both CH3 domains of a respective pair of CH3 domains to produce a cognate (matching) pair, wherein one domain comprises a steric modification of a contact surface in the beta-sheet region that is preferentially attached to the respective contact surface of the other domain through the complementary steric modification. Such steric modifications mainly result from the different amino acid residues and side chains, e.g. to produce a “knob” or “hole” structure, which are complementary to form a “knob into hole” dimer.

According to a specific aspect, each of the CH3 domains in the Fc region is of the IgG type with the amino acid sequence identified as SEQ ID 5 or a functional variant of SEQ ID 5, which is engineered to obtain a strand-exchange by incorporating at least one beta strand IgA segment of at least 2 amino acids length, and the Fc regions preferably comprises a cognate pair of CH3 domains through pairing an IgA segment of the first CH3 domain with an IgA segment of the second CH3 domain. Such strand-exchanged CH3 domains specifically may comprise alternating segments of IgA and IgG amino acid sequences, e.g. incorporating at least 1, 2, 3, 4, or 5 different IgA segments, each located at different positions and separated from each other by a non-IgA segment, e.g. IgG segments.

According to a specific aspect, the ABM is an effector-function competent antibody comprising a Fc gamma receptor binding site and/or a C1q binding site, optionally in the Fc region.

Specifically, the antibody is characterized by any of an ADCC and/or CDC activity.

According to another specific aspect, the ABM is an effector-negative (EN) antibody comprising a Fc region deficient in binding to an Fc gamma receptor and/or C1q.

Specifically, the antibody is effector deficient (herein also referred to as effector negative), with substantially reduced or no binding to an Fc gamma receptor or CD16a via the Fc region.

Specifically, the effector-negative antibody is characterized by a human IgG2 CH2 sequence, or an engineered variant thereof, comprising a modified human IgG2 CH2 domain (F296A, N297Q) described in U.S. Pat. No. 8,562,986, fused to the N-terminus of the C-terminal CH3 domain (numbering according to EU index of Kabat).

Specifically, the EN antibody has a substantially reduced or no ADCC and/or CDC.

Specifically, the ABM comprises an Fc part of an antibody which comprises an FcRn binding site at the interjunction of the CH2 with the CH3 domain, and/or an Fc gamma receptor binding site within the N-terminal region of the CH2 domain, and/or a C1q binding site within the N-terminal region of the CH2 domain.

According to a specific aspect, the ABM comprises a pH-dependent FcRn binding site located in CH2 and/or CH3 domains, if any. Specifically, the FcRn binding site has an affinity to bind the FcRn with a KD of less than 10⁻⁴ M, or less than 10⁻⁵ M, 10⁻⁶ M, 10⁻⁷ M, or 10⁻⁸ M in a pH-dependent manner.

Specifically, the binding affinity to bind FcRn in a pH dependent way is at least 1-log, preferably at least 2-log or 3-log increased at pH5-6 as compared to the same binding affinity at physiological pH (pH7.4).

According to a further aspect, the ABM is engineered to alter the pH dependent FcRn binding. For example, at least one CH3 domain is engineered to comprise at least one mutation at the FcRn binding site to reduce pH-dependent FcRn binding, specifically at least one of the H433A or H435A mutations, or both H433A and H435A mutations, wherein the numbering is according to the EU index of Kabat. Reduction of pH-dependent FcRn binding may be such that the binding affinity to bind FcRn in a pH dependent way is less than 1-log, preferably about the same or less at pH5-6 as compared to the same binding affinity at physiological pH (pH7.4).

Specific embodiments refer to any of the ABM exemplified herein, or comprising any of the heavy and light chains or any of the pairs of heavy and light chains described in the Examples section. Specifically, an ABM as described herein may comprise or consist of the heavy and light chains described in the Examples section.

Specifically, the ABM described herein is provided for medical, diagnostic or analytical use.

The invention further provides for a pharmaceutical preparation comprising the ABM described herein, preferably in a parenteral or mucosal formulation, optionally containing a pharmaceutically acceptable carrier or excipient.

The invention further provides for an isolated nucleic acid encoding an ABM described herein.

The invention further provides for an expression cassette or a plasmid comprising or incorporating the nucleic acid described herein and optionally further sequences to express the ABM encoded by the nucleic acid sequence, such as regulatory sequences.

Specifically, the expression cassette or the plasmid comprises a coding sequence to express the ABM described herein, or the HC and/or LC of the ABM described herein.

According to a specific example, the ABM consists of one or more HCs and LCs, wherein each of the HCs is characterized by the same HC amino acid sequence, and each of the LCs is characterized by the same LC amino acid sequence, and the coding sequences for the HC and the LC are employed to produce a monovalent or homodimeric antibody.

According to another specific example, the ABM consists of two different HCs and two different LCs, and the coding sequences for the two different HCs and the two different LCs are employed to produce a heterodimeric or bispecific antibody.

The invention further provides for a production host cell comprising at least one expression cassette or a plasmid incorporating one or more nucleic acid molecules encoding an ABM described herein.

Specifically, the host cell transiently or stably expresses the ABM. According to specific examples, the host cell is a eukarytoc host cell, preferably any of yeast or mammalian cells.

The invention further provides for a method of producing an ABM described herein, wherein a host cell according described herein is cultivated or maintained under conditions to produce said ABM.

Specifically, the ABM may be isolated and/or purified from the cell culture supernatant. According to a specific example, the ABM is a bispecific full-length antibody which is heterodimeric comprising two different HCs and two different LCs, and the ABM comprises a correct pairing of the cognate HC/LC pairs and cognate CL and CH1 domains, respectively, and the ABM is produced by the host cell, wherein less than 10% of the antibodies produced are incorrectly paired, preferably less than 5%, as measured by mass spectrometry (LC-ESI-MS) compairing maximum peak intensity.

### FIGURES

FIG. 1: The bispecific IgG BxM was produced transiently in Expi293F either carrying no interface mutations (left panel) or carrying the interface mutations of MaB40 (right panel). Both antibodies were deglycosylated and analysed by LC-ESI-MS. B10v5 light and heavy chain are shown in white and hu225M light and heavy chain are shown in black. The relative abundance of each detected chain pairing variant is indicated as a percentage of all detected complete IgGs. In BxM wt both variants with mispairing in the Fab are detectable in considerable amounts (12% each when compairing maximum peak intensity). Hence, the peak of the correctly paired variant will also contain the mispaired variant where the light chains have swapped positions. The production of BxM MaB40 yielded only the correctly paired variant. Mispaired variants disappeared due to the interface engineering.

FIG. 2: Analytical size exclusion chromatography of purified BxM wildtype and BxM MaB40. Both IgGs elute at the expected time of 16.3 min. No negative impact of the interface engineering on the SEC profile was detectable.

FIG. 3: The bispecific IgG BxO was produced transiently in HEK293-6E either carrying no interface mutations (BxO wt, upper left panel) or carrying the interface mutations of MaB40 (BxO MaB40, upper right panel). Supportive mutations in the B10v5 Fab were introduced which led to the creation of the bispecific antibodies BxO MaB5/40, BxO MaB21/40 and BxO MaB45/40 (remaining lower panels). All antibodies were deglycosylated and analysed by LC-ESI-MS. B10v5 light and heavy chain are shown in white and OKT3 light and heavy chain are shown in black. The relative abundance of each detected chain pairing variant is indicated as a percentage of all detected complete IgGs. In BxO wt both variants with mispairing in the Fab are detectable in varying amounts, accumulating to more than 40% of mispaired antibody. Mispairing was reduced considerably in BxO MaB40 but still detectable. BxO containing not only the mutations of MaB40 but also either of the supportive mutations showed improved pairing behaviour. More than 90% of all detected complete IgGs were correctly paired BxO.

FIG. 4: Analytical size exclusion chromatography of purified BxO wildtype, BxO MaB40, BxO MaB5/40 and BxO MaB45/40. All IgGs elute at the expected time of 15.4 min. No negative impact of the interface engineering on the SEC profile was detectable.

FIG. 5: Sequences

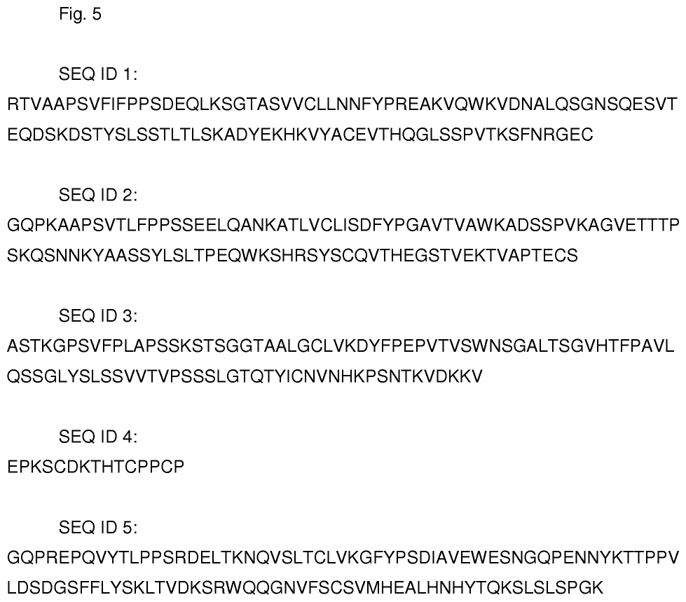

SEQ ID 1: amino acid sequence of a Ckappa domain of human IgG1

SEQ ID 2: amino acid sequence of a Clambda domain of human IgG1

SEQ ID 3: amino acid sequence of a CH1 domain of human IgG1

SEQ ID 4: amino acid sequence of a human IgG1 hinge region

SEQ ID 5: amino acid sequence of a CH3 domain of human IgG1

### DETAILED DESCRIPTION

Specific terms as used throughout the specification have the following meaning.

The term “antigen-binding molecule” or ABM as used herein shall mean a molecule comprising a binding domain which is a polypeptide that specifically recognizes or binds to an antigen or epitope thereof with a certain binding affinity and/or avidity. According to specific examples of an ABM the binding domain is an immunoglobulin-type binding region comprising a polypeptide selected from the group consisting of a single-domain antibody, single-chain variable domains, Fd fragment, Armadillo repeat polypeptide, fibronectin type III domain, tenascin type III domain, ankyrin repeat motif domain, lipocalin, Kunitz domain, Fyn-derived SH2 domain, miniprotein, C-type lectin-like domain scaffold, engineered antibody mimic, and any genetically manipulated counterparts of any of the foregoing which retain antigen binding functionality.

Specific embodiments of an ABM comprise or consist of an antibody or antigen-binding fragment thereof.

The term “antibody” as used herein is defined as antigen-binding polypeptides that are either immunoglobulins or immunoglobulin-like molecules, or other proteins exhibiting modular antibody formats, e.g. composed of one or more antibody domains and bearing antigen-binding properties similar to immunoglobulins or antibodies, in particular proteins that may exhibit mono- or bi- or multi-specific, or mono-, bi- or multivalent binding properties, e.g. at least two specific binding sites for epitopes of e.g. antigens, effector molecules or structures, specifically of pathogen origin or of human structure, like self-antigens including cell-associated or serum proteins. The terms “antibody” and “immunoglobulin” are herein used interchangeably.

An antibody typically consists of or comprises antibody domains, which are understood as constant and/or variable domains of the heavy and/or light chains of immunoglobulins, with one or more or without a linker sequence. Antibodies are specifically understood to consist of or comprise combinations of variable and/or constant antibody domains with or without a linking sequence or hinge region, including pairs of variable antibody domains, such as one or two VH/VL pairs. Polypeptides are understood as antibody domains, if comprising a beta-barrel structure consisting of at least two beta-strands of an antibody domain structure connected by a loop sequence. Antibody domains may be of native structure or modified by mutagenesis or derivatization, e.g. to modify the antigen binding properties or any other property, such as stability or functional properties, such as binding to the Fc receptors FcRn and/or Fcgamma receptor.

The term “antibody” as used herein specifically includes full-length antibodies, including antibodies of immunoglobulin-like structures. Specifically, an antibody can be a full-length antibody, e.g. of an IgG type (e.g., an IgG1, IgG2, IgG3, or IgG4 subtype), IgA1, IgA2, IgD, IgE, or IgM antibody.

The term further includes any of derivatives, combinations or fusions of antibodies, antibody domains, or antibody fragments.

The term “full length antibody” is used to refer to any antibody molecule comprising an Fc region or at least most of the Fc part of an antibody, which specifically includes a dimer of heavy chains. This term “full length antibody” is used herein to emphasize that a particular antibody molecule is not an antibody fragment.

In accordance therewith, an antibody is typically understood as a protein (or protein complex) that includes one or more polypeptides substantially encoded by immunoglobulin genes or fragments of immunoglobulin genes. The recognized immunoglobulin genes include the kappa, lambda, alpha, gamma, delta, epsilon, and mu constant region genes, as well as immunoglobulin variable region genes. Light chains (LC) are classified as either kappa (including a VL and a Clambda domain) or lambda (including a VL and a Ckappa domain). Heavy chains (HC) are classified as gamma, mu, alpha, delta, or epsilon, which in turn define the immunoglobulin classes, IgG, IgM, IgA, IgD and IgE, respectively.

A HC or LC is each composed of at least two domains connected to each other to produce a chain of domains. It is specifically understood that an antibody HC includes a VH antibody domain and at least one antibody domain C-terminally bound to the VH, i.e. the at least one antibody domain is connected to the C-terminus of the VH domain with or without a linking sequence. An antibody LC includes a VL antibody domain and at least one antibody domain C-terminally bound to the VL, i.e. the at least one antibody domain is connected to the C-terminus of the VL domain with or without a linking sequence.

The definition further includes domains of the heavy and light chains of the variable region (such as dAb, Fd, VI, Vk, Vh, VHH) and the constant region or individual domains of an intact antibody such as CH1, CH2, CH3, CH4, Cl and Ck, as well as mini-domains consisting of at least two beta-strands of an antibody domain connected by a structural loop. Typically, an antibody having an antigen-binding site through a specific CDR structure is able to bind a target antigen through the CDR loops of a pair of VH/VL domains.

The term “antibody” shall specifically include antibodies in the isolated form, which are substantially free of other antibodies directed against different target antigens and/or comprising a different structural arrangement of antibody domains. Still, an isolated antibody may be comprised in a combination preparation, containing a combination of the isolated antibody, e.g. with at least one other antibody, such as monoclonal antibodies or antibody fragments having different specificities.

The term “antibody” shall apply to antibodies of animal origin, including human species, such as mammalian, including human, murine, rabbit, goat, camelid, llama, cow and horse, or avian, such as hen, which term shall particularly include recombinant antibodies which are based on a sequence of animal origin, e.g. human sequences.

The term “antibody” specifically applies to human antibodies.

The term “human” as used with respect to an antibody is understood to include antibodies having variable and constant regions derived from human germline immunoglobulin sequences. A human antibody may include amino acid residues not encoded by human germline immunoglobulin sequences (e.g., mutations introduced by random or site-specific mutagenesis in vitro or by somatic mutation in vivo), for example in the CDRs. Human antibodies include antibodies isolated from human immunoglobulin libraries or from animals transgenic for one or more human immunoglobulin.

A human antibody is preferably selected or derived from the group consisting of IgA1, IgA2, IgD, IgE, IgG1, IgG2, IgG3, IgG4 and IgM.

A murine antibody is preferably selected or derived from the group consisting of IgA, IgD, IgE, IgG1, IgG2A, IgG2B, IgG2C, IgG3 and IgM.

The term “antibody” further applies to chimeric antibodies, e.g. chimeric antibodies, with sequences of origin of different species, such as sequences of murine and human origin.

The term “chimeric” as used with respect to an antibody refers to those molecules wherein one portion of each of the amino acid sequences of heavy and light chains is homologous to corresponding sequences in immunoglobulins derived from a particular species or belonging to a particular class, while the remaining segment of the chain is homologous to corresponding sequences in another species or class. Typically the variable region of both light and heavy chains mimics the variable regions of immunoglobulins derived from one species of mammals, while the constant portions are homologous to sequences of immunoglobulins derived from another. For example, the variable region can be derived from presently known sources using readily available B-cells or hybridomas from non-human host organisms in combination with constant regions derived from, for example, human cell preparations.

The term “antibody” may further apply to humanized antibodies.

The term “humanized” as used with respect to an antibody refers to a molecule having an antigen binding site that is substantially derived from an immunoglobulin from a non-human species, wherein the remaining immunoglobulin structure of the molecule is based upon the structure and/or sequence of a human immunoglobulin. The antigen binding site may either comprise complete variable domains fused onto constant domains or only the complementarity determining regions (CDR) grafted onto appropriate framework regions in the variable domains. Antigen-binding sites may be wild-type or modified, e.g. by one or more amino acid substitutions, preferably modified to resemble human immunoglobulins more closely. Some forms of humanized immunoglobulins preserve all CDR sequences (for example a humanized mouse antibody which contains all six CDRs from the mouse antibody). Other forms have one or more CDRs which are altered with respect to the original antibody.

According to a specific embodiment, all antibody domains comprised in the ABM as described herein are of human origin or humanized or functionally active variants thereof with at least 60% sequence identity, or at least 70%, 80%, 90%, or 95% sequence identity, preferably wherein the origin of the antibody domains is any of an IgG1, IgG2, IgG3, IgG4, IgA, IgM, or IgE antibody. Specifically, all antibody domains originate from the same basic immunglobulin fold, although b-sheet formats may differ, and connecting loops certainly be variable, especially in V domains.

The term “antibody” further applies to monoclonal or polyclonal antibodies, specifically a recombinant antibody, which term includes all antibodies and antibody structures that are prepared, expressed, created or isolated by recombinant means, such as antibodies originating from animals, e.g. mammalians including human, that comprises genes or sequences from different origin, e.g. chimeric, humanized antibodies, or hybridoma derived antibodies. Further examples refer to antibodies isolated from a host cell transformed to express the antibody, or antibodies isolated from a recombinant, combinatorial library of antibodies or antibody domains, or antibodies prepared, expressed, created or isolated by any other means that involve splicing of antibody gene sequences to other DNA sequences.

The term “antibody” is understood to include functionally active variants of new or existing, e.g. naturally-occurring antibodies. It is further understood that the term variant of an antibody, in particular variants of antibody-like molecules, or antibody variants, shall also include derivatives of such molecules as well.

A derivative is any combination of one or more antibodies and or a fusion protein in which any domain or minidomain of the antibody may be fused at any position to one or more other proteins, such as to other antibodies or antibody fragments, but also to ligands, enzymes, toxins and the like. The ABM or antibody described herein can specifically be used as isolated polypeptides or as combination molecules, e.g. through recombination, fusion or conjugation techniques, with other peptides or polypeptides. The peptides are preferably homologous to antibody domain sequences, and are preferably at least 5 amino acids long, more preferably at least 10 or even at least 50 or 100 amino acids long, and constitute at least partially the loop region of the antibody domain.

A derivative of an antibody may also be obtained by association or binding to other substances by various chemical techniques such as covalent coupling, electrostatic interaction, di-sulphide bonding etc. The other substances bound to the antibodies may be lipids, carbohydrates, nucleic acids, organic and inorganic molecules or any combination thereof (e.g. PEG, prodrugs or drugs). A derivative would also comprise an antibody with the same amino acid sequence but made completely or partly from non-natural or chemically modified amino acids. In a specific embodiment, the antibody is a derivative comprising an additional tag allowing specific interaction with a biologically acceptable compound. There is not a specific limitation with respect to the tag usable, as far as it has no or tolerable negative impact on the binding of the antibody to its target. Examples of suitable tags include His-tag, Myc-tag, FLAG-tag, Strep-tag, Calmodulin-tag, GST-tag, MBP-tag, and S-tag. In another specific embodiment, the antibody is a derivative comprising a label. The term “label” as used herein refers to a detectable compound or composition which is conjugated directly or indirectly to the antibody so as to generate a “labeled” antibody. The label may be detectable by itself, e.g. radioisotope labels or fluorescent labels, or, in the case of an enzymatic label, may catalyze chemical alteration of a substrate compound or composition which is detectable.

A derivative of an antibody is e.g. derived from a parent antibody or antibody sequence, such as a parent antigen-binding (e.g. CDR) or framework (FR) sequence, e.g. mutants or variants obtained by e.g. in silico or recombinant engineering or else by chemical derivatization or synthesis.

The term “variants” as used herein shall specifically include any “mutant”, “homolog”, or “derivative” as described herein. The term “variant” shall specifically encompass functionally active variants which are characterized by a certain functionality.

The functionality of the ABM or the antibody described herein is particularly characterized by a certain antigen-binding property (in particular the epitope specificity) and the preferred pairing of the CL and CH1 domains, wherein the amino acid at the position 18 in the CL domain and/or the amino acid at the position 26 in the CH1 domain are characterized by opposite polarity (numbering is according to the IMGT). The functionality of functional variants of antibody constant domains is herein understood as the capability of pairing with the counterpart antibody domain to produce an antibody domain pair. In particular, the functional variants of the CL and CH1 domains described herein comprise the dominant point mutations for preferred pairing, wherein the amino acid at the position 18 in the CL domain and/or the amino acid at the position 26 in the CH1 domain are characterized by opposite polarity (“dominant point mutations”; numbering according to the IMGT), and optionally further point mutations which support the preferred pairing to produce the CL/CH1 dimer, but do not decrease the likelihood of pairing.

The term “variant” shall particularly refer to antibodies, such as mutant antibodies or fragments of antibodies, e.g. obtained by mutagenesis methods, in particular to delete, exchange, introduce inserts into a specific antibody amino acid sequence or region or chemically derivatize an amino acid sequence, e.g. in the constant domains to engineer the antibody stability, effector function or half-life, or in the variable domains to improve antigen-binding properties, e.g. by affinity maturation techniques available in the art. Any of the known mutagenesis methods may be employed, including point mutations at desired positions, e.g. obtained by randomization techniques. In some cases positions are chosen randomly, e.g. with either any of the possible amino acids or a selection of preferred amino acids to randomize the antibody sequences. The term “mutagenesis” refers to any art recognized technique for altering a polynucleotide or polypeptide sequence. Preferred types of mutagenesis include error prone PCR mutagenesis, saturation mutagenesis, or other site directed mutagenesis.

The term “functional variants” herein also referred to as “functionally active variant” may e.g. include a sequence resulting from modification of a parent sequence (e.g. from a a parent antibody) by insertion, deletion or substitution of one or more amino acids, or chemical derivatization of one or more amino acid residues in the amino acid sequence, or nucleotides within the nucleotide sequence, or at either or both of the distal ends of the sequence, e.g. in a CDR or FR sequence, and which modification does not affect, in particular impair, the activity of this sequence. In the case of a binding site having specificity to a selected target antigen, the functionally active variant of an antibody would still have the predetermined binding specificity, though this could be changed, e.g. to change the fine specificity to a specific epitope, the affinity, the avidity, the Kon or Koff rate, etc. For example, an affinity matured antibody is specifically understood as a functionally active variant antibody. Hence, the modified CDR sequence in an affinity matured antibody is understood as a functionally active variant.

The functional activity is preferably determined by the structure and function of the variant as compared to a parent molecule, e.g. in an assay for determining the specificity of binding a target antigen and/or the required in vivo half-life of the molecule and/or the FcRn binding in a pH dependent way, e.g., determined in a standard assay by measuring functionality of the antibody.

The functional activity of an antibody in terms of antigen-binding is typically determined in an ELISA assay, BIAcore assay, Octet BLI assay, or FACS based assay when the antigen is expressed on cell surface.

Functionally active variants may be obtained, e.g. by changing the sequence of a parent antibody, e.g. a monoclonal antibody having a specific native structure of an antibody, such as an IgG1 structure, to obtain a variant having the same specificity in recognizing a target antigen, but having a structure which differs from the parent structure, e.g. to modify any of the antibody domains to introduce specific mutations, to produce bispecific constructs, or to produce a fragment of the parent molecule.

Typically, a parent antibody or sequence may be modified to produce variants which incorporate mutations within a sequence region besides the antigen-binding site, or within the binding site, that does not impair the antigen binding, and preferably would have a biological activity similar to the parent antibody, including the ability to bind an antigen, e.g. with substantially the same biological activity, as determined by a specific binding assay or functional test to target the antigen.

The term “substantially the same biological activity” as used herein refers to the activity as indicated by substantially the same activity being at least 20%, at least 50%, at least 75%, at least 90%, e.g. at least 100%, or at least 125%, or at least 150%, or at least 175%, or e.g. up to 200% of the activity as determined for the comparable or parent antibody.

The preferred variants as described herein are functionally active with regard to the antigen binding, preferably which have a potency to specifically bind the individual antigen, and not significantly binding to other antigens that are not target antigens, e.g. with a Kd value difference of at least 2 logs, preferably at least 3 logs. The antigen binding by a functionally active variant is typically not impaired, corresponding to about substantially the same binding affinity as the parent antibody or sequence, or antibody comprising a sequence variant, e.g. with a a Kd value difference of less than 2 logs, preferably less than 3 logs, however, with the possibility of even improved affinity, e.g. with a Kd value difference of at least 1 log, preferably at least 2 logs.

In a preferred embodiment the functionally active variant of a parent antibody

a) is a biologically active fragment of the antibody, the fragment comprising at least 50% of the sequence of the molecule, preferably at least 60%, at least 70%, at least 80%, at least 90%, or at least 95% and most preferably at least 97%, 98% or 99%;

b) is derived from the antibody by at least one amino acid substitution, addition and/or deletion, wherein the functionally active variant has a sequence identity to the molecule or part of it, such as an antibody of at least 50% sequence identity, preferably at least 60%, more preferably at least 70%, more preferably at least 80%, still more preferably at least 90%, even more preferably at least 95% and most preferably at least 97%, 98% or 99%; and/or

c) consists of the antibody or a functionally active variant thereof and additionally at least one amino acid or nucleotide heterologous to the polypeptide or the nucleotide sequence.

In one embodiment, the functionally active variant of the ABM or antibody as described herein is essentially identical to the variant described above, but differs from its polypeptide or the encoding nucleotide sequence, respectively, in that it is derived from a homologous sequence of a different species. These are referred to as naturally occurring variants or analogs.

The term “functionally active variant” also includes naturally occurring allelic variants, as well as mutants or any other non-naturally occurring variants. As is known in the art, an allelic variant is an alternate form of a (poly) peptide that is characterized as having a substitution, deletion, or addition of one or more amino acids that does essentially not alter the biological function of the polypeptide.

Functionally active variants may be obtained by sequence alterations in the polypeptide or the nucleotide sequence, e.g. by one or more point mutations, wherein the sequence alterations retains or improves a function of the unaltered polypeptide or the nucleotide sequence, when used as described herein. Such sequence alterations can include, but are not limited to, (conservative) substitutions, additions, deletions, mutations and insertions.

Specific functionally active variants are CDR variants. A CDR variant includes an amino acid sequence modified by at least one amino acid in the CDR region, wherein said modification can be a chemical or a partial alteration of the amino acid sequence, which modification permits the variant to retain the biological characteristics of the unmodified sequence. A partial alteration of the CDR amino acid sequence may be by deletion or substitution of one to several amino acids, e.g. 1, 2, 3, 4 or 5 amino acids, or by addition or insertion of one to several amino acids, e.g. 1, 2, 3, 4 or 5 amino acids, or by a chemical derivatization of one to several amino acids, e.g. 1, 2, 3, 4 or 5 amino acids, or combination thereof. The substitutions in amino acid residues may be conservative substitutions, for example, substituting one hydrophobic amino acid for an alternative hydrophobic amino acid.

Conservative substitutions are those that take place within a family of amino acids that are related in their side chains and chemical properties. Examples of such families are amino acids with basic side chains, with acidic side chains, with non-polar aliphatic side chains, with non-polar aromatic side chains, with uncharged polar side chains, with small side chains, with large side chains etc.

A point mutation is particularly understood as the engineering of a polynucleotide that results in the expression of an amino acid sequence that differs from the non-engineered amino acid sequence in the substitution or exchange, deletion or insertion of one or more single (non-consecutive) or doublets of amino acids for different amino acids.

According to a certain aspect, the point mutations of the CL and CH1 domain as described herein for the preferred pairing of the antibody CL and CH1 domains are changing the polarity of the amino acid residues at the position 18 in the CL domain and/or the amino acid at the position 26 in the CH1 domain to the opposite polarity, wherein numbering is according to the IMGT. Specific embodiments refer to the following:

a) where the amino acid residues at the position 18 in the CL domain is of positive polarity, the amino acid residue at the position 26 in the CH1 domain is of negative polarity; or

b) where the amino acid residues at the position 18 in the CL domain is of negative polarity, the amino acid residue at the position 26 in the CH1 domain is of positive polarity.

The above described point mutations are herein referred to as “dominant” point mutations, because by such point mutations of opposite polarity in the indicated positions, ABM or antibodies can be produced which are characterized by the preferred pairing of the CL and CH1 domains bearing such point mutations, even if there are no further point mutations in the CL or CH1 domains, or in any of the adjacent VL or VH domains.

Besides the dominant point mutations, there may be further point mutations, which even improve the preferred pairing of the LC and the HC, for example point mutations which are herein referred to as “supportive” point mutations. Such supportive point mutations may be engineered in any of the CL and/or counterpart CH1 domains, or in the VL and/or counterpart VH domains. Exemplary supportive point mutations are the following: point mutation F7X in the CL domain, wherein X is any of S, A, or V; and point mutation A20L in the counterpart CH1 domain, wherein numbering is according to the IMGT. Typically, the supportive point mutations are conservative point mutations, characterized by a substitution of amino acid residues, wherein the polarity of the amino acid residues is not changed by such substitution.

Variants of the ABM or antibody as described herein may include point mutations which refer to the exchange of amino acids of the same polarity and/or charge. In this regard, amino acids refer to 20 naturally-occurring amino acids encoded by sixty-four triplet codons. These 20 amino acids can be split into those that have neutral charges, positive charges, and negative charges:

The 20 naturally-occurring amino acids are shown in the table below along with their respective three-letter and single-letter code and polarity:

| | 3- | 1- | |
|---|---|---|---|
| Amino-acid | letter | letter | |
| name | code | code | Properties |
| Alanine | Ala | A | Non-polar; Hydrophobic |
| Arginine | Arg | R | Positively charged (basic amino acids; non-acidic amino |
| | | | acids); Polar; Hydrophilic; pK = 12.5 |
| Asparagine | Asn | N | No charge (non-acidic amino acids); Polar; Hydrophilic |
| Aspartate | Asp | D | Negatively charged (acidic amino acids); Polar; |
| | | | Hydrophilic; pK = 3.9 |
| Cysteine | Cys | C | No charge (non-acidic amino acids); Non-polar; |
| | | | Hydrophilic |
| Glutamate | Glu | E | Negatively charged (acidic amino acids); Polar; |
| | | | Hydrophilic; pK = 4.2 |
| Glutamine | Gln | Q | No charge (non-acidic amino acids); Polar; Hydrophilic |
| Glycine | Gly | G | No charge (non-acidic amino acids); Non-polar; |
| | | | Hydrophilic |
| Histidine | His | H | Positively charged (basic amino acids; non-acidic amino |
| | | | acids); Polar; Hydrophilic; pK = 6.0 |
| Isoleucine | Ile | I | Non-polar; Hydrophobic |
| Leucine | Leu | L | Non-polar; Hydrophobic |
| Lysine | Lys | K | Positively charged (basic amino acids; non-acidic amino |
| | | | acids); Polar; Hydrophilic; pK = 10.5 |
| Methionine | Met | M | Non-polar; Hydrophobic |
| Phenylalanine | Phe | F | Non-polar; Hydrophobic |
| Proline | Pro | P | Non-polar; Hydrophobic |
| Serine | Ser | S | No charge (non-acidic amino acids); Polar; Hydrophilic |
| Threonine | Thr | T | No charge (non-acidic amino acids); Polar; Hydrophilic |
| Tryptophan | Trp | W | No charge; Non-polar; Hydrophobic |
| Tyrosine | Tyr | Y | No charge (non-acidic amino acids); Polar; Hydrophilic |
| Valine | Val | V | Non-polar; Hydrophobic |

“Percent (%) amino acid sequence identity” with respect to polypeptide sequences is defined as the percentage of amino acid residues in a candidate sequence that are identical with the amino acid residues in the specific polypeptide sequence, after aligning the sequence and introducing gaps, if necessary, to achieve the maximum percent sequence identity, and not considering any conservative substitutions as part of the sequence identity. Those skilled in the art can determine appropriate parameters for measuring alignment, including any algorithms needed to achieve maximal alignment over the full length of the sequences being compared.

An ABM or antibody variant is specifically understood to include homologs, analogs, fragments, modifications or variants with a specific glycosylation pattern, e.g. produced by glycoengineering, which are functional and may serve as functional equivalents, e.g. binding to the specific targets and with functional properties. An ABM or antibody may be glycosylated or unglycosylated. For example, a recombinant ABM or antibody as described herein may be expressed in an appropriate mammalian cell to allow a specific glycosylation of the molecule as determined by the host cell expressing the antibody.

The term “beta-sheet” or “beta strand” of an antibody domain, in particular of a constant antibody domain such as a CL or CH1 domain is herein understood in the following way. An antibody domain typically consists of at least two beta strands connected laterally by at least two or three backbone hydrogen bonds, forming a generally twisted, pleated sheet. A beta strand is a single continuous stretch of amino acids of typically 3 to 10 amino acids length adopting such an extended conformation and involved in backbone hydrogen bonds to at least one other strand, so that they form a beta sheet. In the beta sheet, the majority of beta strands are arranged adjacent to other strands and form an extensive hydrogen bond network with their neighbors in which the N-H groups in the backbone of one strand establish hydrogen bonds with the C=O groups in the backbone of the adjacent strands.

The structure of antibody constant domains, such as CL or CH1 domains, is similar to that of variable domains, consisting of beta-strands connected by loops, some of which contain short alpha-helical stretches. The framework is mostly rigid and the loops are comparatively more flexible, as can be seen from the b-factors of various Fc crystal structures. An antibody CL or CH1 domain typically has seven beta strands forming a beta-sheet (A-B-C-D-E-F-G), wherein the beta strands are linked via loops, three loops being located at the N-terminal tip of the domain (A-B, C-D, E-F), and further three loops being located at the N-terminal tip of the domain (B-C, D-E, F-G). A “loop region” of a domain refers to the portion of the protein located between regions of beta strands (for example, each of the CL or CH1 domains comprises seven beta sheets, A to G, oriented from the N- to C-terminus).

Preferably a pair of antibody domains, such as the pair of CL and CH1 domains that produces a (hetero)dimer by connecting a binding surface involving the A, B and/or E strands of each of the domains (herein referred to as binding interface). By such contact of the beta-sheet region of the CL domain with the beta-sheet region of the CH1 domain, a dimer (designated as CL/CH1) is produced.

Specifically, the CL and CH1 domains as described herein comprise or consist of an amino acid sequence of a human IgG1 antibody.

In particular, the Ckappa domain is characterized by the amino acid sequence identified as SEQ ID 1, or a functional variant thereof, e.g. with a certain sequence identity.

In particular, the Clambda domain is characterized by the amino acid sequence identified as SEQ ID 2, or a functional variant thereof, e.g. with a certain sequence identity.

In particular, the CH1 domain is characterized by the amino acid sequence identified as SEQ ID 3, or a functional variant thereof, e.g. with a certain sequence identity.

Alternatively, the CL and CH1 antibody domains as described herein comprise or consist of an amino acid sequence of any of a human IgG2, IgG3, IgG4, IgA, IgM, IgE, IgD, or a functional variant thereof, e.g. with a certain sequence identity.

The Fv part of an antibody is typically understood as the pair of VL and VH domains that produces a (hetero)dimer by connecting a binding surface involving the C, C′ and F strands of each of the domains (the binding interface). By such contact of the beta-sheet region of the VL domain with the beta-sheet region of the VH domain, a dimer (designated as VL/VH) is produced.

A Fab arm is herein understood as the pair of a first and a second antibody chain, wherein the first chain comprises or consists of a VL domain and a CL domain, which is linked to the C-terminus of the VL domain (light chain, LC), and the second chain comprises or consists of a VH domain and a CH1 domain, which is linked to the C-terminus of the VH domain (heavy chain, HC), wherein the VL connects to (pairs with) the VH via the binding interface, and the CL connects to (pairs with) the CH1 via the binding interface, thereby producing a (hetero)dimer of the LC and HC (also designated LC/HC).

The Fc part of an antibody is herein understood as the pair of antibody chains, each comprising a CH2 domain and a CH3 domain, which is linked to the C-terminus of the CH2 domain (Fc chains), wherein the CH2 domains of each of the antibody chains connect to each other via the binding surface involving the A, B and/or E strands of each of the CH2 domains (the binding interface), and wherein the CH3 domains of each of the antibody chains connect to (pair with) each other via the binding surface involving the A, B and/or E strands of each of the CH3 domains (the binding interface), thereby producing a (homo)dimer of Fc chains. The Fc part described herein can be from an IgG, IgA, IgD, IgE or IgM.

In one embodiment described herein, the Fc part comprises mutated CH3 domains, e.g. which have at least a portion of one or more beta strands replaced with heterologous sequences, such as to include one or more point mutations, or knob or hole mutations. In such case the Fc region comprises a heterodimer of the Fc chains, characterized by the assembly of two different CH3 domains.

Specific knob mutations are one or more amino acid substitutions to increase the contact surface between two domains by incorporating one or more amino acids which provide for an additional protuberance of a beta-strand structure, e.g. one or more of CH3 knob mutations selected from the group consisting of T366Y, T366W, T394W, F405A. A specific knob modification denotes the mutation T366W in the CH3 domain of an antibody (numbering according to EU index of Kabat). Knob mutations specifically provide a matching (cognate) surface to bind another antibody domain, e.g. which is modified to incorporate hole mutations.

Specific hole mutations are one or more amino acid substitutions to increase the contact surface between two domains by incorporating one or more amino acids which provide for an additional cave of a beta-strand structure, e.g. one or more of CH3 hole mutations selected from the group consisting T366S, L368A and Y407V. A specific hole-modification denotes any of the mutations T366S, L368A, Y407V, Y407T in the CH3 domain of an antibody (numbering according to EU index of Kabat). Hole mutations specifically provide a matching (cognate) surface to bind another antibody domain, e.g. which is modified to incorporate knob mutations.

Matching knob into hole mutations are, e.g. T366Y on one CH3 domain and the matching Y407′T on the second CH3 domain of the CH3 domain pair, herein referred to as T366Y/Y407′T. Further matching mutations are

T366Y/Y407′T,

F405A/T394′W,

T366Y:F405A/T394W:Y407′T,

T366W/Y407′A, and/or

S354C:T366W/Y349′C:T366′S:L368′A:Y407V.

Specific CH3 mutations include an intermolecular beta-strand swap, e.g. wherein one or more segments or sequences within a CH3 beta strand are mutated to incorporate segments or sequences of antibody domains which differ from the original CH3 domain, e.g. of antibody domains of a different type or subtype. Specific mutants are obtained by strand exchange, wherein a CH3 domain of an IgG type incorporates one or more segments or sequences of a CH3 domain of an IgA type. If two strand exchanged CH3 domains are mutated to form a cognate pair, the IgA segments or sequences of each of the CH3 domains produce an interdomain contact surface which is cognate, such that the mutated CH3 domains preferentially pair with each other over a wild-type CH3 domain. Specific examples of such modifications of antibody domains to incorporate a segment swap may be strand-exchange engineered domains (SEED). Such modifications may be used to produce asymmetric or bispecific antibodies by preferentially pairing the SEED modified CH3 domains of the heavy chains. This is based on exchanging structurally related sequences within the conserved CH3 domains. Alternating sequences from human IgA and IgG in the SEED CH3 domains generate two asymmetric but complementary domains, designated AG and GA. The SEED design allows efficient generation of AG/GA heterodimers, while disfavoring homodimerization of AG and GA SEED CH3 domains.

The connection of antibody domains or LC/HC, or Fc chains may be further supported by intradomain or interdomain disulfide bridges. Disulfide bonds are usually formed from the oxidation of thiol groups of two cysteins, thereby linking the S-atoms to form a disulfide bridge between the two cysteine residues.

According to a specific embodiment, antibody domains include mutations incorporating cysteine residues which are capable of forming disulfide bridges to stabilize an antibody domain by an additional intradomain disulfide bridge, or a pair of antibody domains by an additional interdomain disulfide bridge. Specifically, cysteine may be inserted (by an additional amino acid or an amino acid substitution) in the C-terminal region or at the C-terminus of a CH3 domain. A pair of CH3 that bears an additional cysteine modification can be stabilized by disulfide bond formation between the CH3 pair, thereby producing a CH3/CH3 dimer. In some embodiments disulfide-linked antibody domains are homodimers or heterodimers, thus, pairs of the same or different domains.

In order to allow proper pairing of antibody chains or domains, any of the CH3 mutations may specifically be employed, e.g. the knobs-into-holes technology, the SEED technology, charge repulsion technology, disulfide linkage or the cross-mAb technology can be used in order to reduce the amount of not correctly associated molecules.

A “pair” of antibody domains is herein understood as a set of two antibody domains, where one has an area on its surface or in a cavity that specifically binds to, and is therefore complementary to, an area on the other one. Antibody domains may associate and assemble to form a pair of antibody domains through contact of a beta-sheet region. Such domain pair is also referred to as a dimer, which is e.g. associated by electrostatic interaction, recombinant fusion or covalent linkage, placing two domains in direct physical association, e.g. including both in solid and in liquid form. Specifically described herein is a CL/CH1 dimer which can be a preferred pair of cognate antibody domains through certain point mutations at positions identified herein.

“Preferred pairing” is herein understood as the formation of dimers of antibody domains or antibody chains thereby obtaining pairs of antibody domains or antibody chains, wherein the pair is formed through an increased affinity or avidity of the binding interfaces of the antibody domains and an increased (thermo-) stability of the domain pair or the HC/LC pair. Cognate antibody domains may be produced by modifications in the interface region, such as described herein, which preferably pair with each other over any wild-type domain of the same type.

In a pair of antibody domains the antibody domains are herein referred to as “counterpart” domains. In an antibody described herein the following domains are considered counterparts suitably forming a pair of antibody domains (counterparts separated by a slash (/)):

VL/VH;

CL (Clambda or Ckappa)/CH1;

CH2/CH2;

CH3/CH3.

The term “cognate” with respect to a pair of domains or domain dimer is understood as domains which have a matching binding point or structure to obtain a contact surface on each of the domains to preferentially form a pair of such domains. Specific domains are understood as “cognate” or a cognate pair of domains, if at least one of the domains is modified to preferentially bind its cognate (counterpart) binding partner to produce the domain pair. Preferably, both cognate domains are engineered to incorporate matching mutations, e.g. mutations to introduce amino acid residues of opposite polarities, knob-into-hole mutations, SEED mutations, additional cysteine residues for disulfide bridge formation, or modifications employing charge repulsion technology.

The term “multivalent” with respect to an ABM or antibody as described herein shall refer to a molecule having at least two binding sites to bind the same target antigen, specifically binding the same or different epitopes of such target antigen. The term shall include bivalent antibodies or molecules with 2 or more valencies to bind the target antigen, e.g. through at least 2, 3, 4 or even more binding sites. For example, a bivalent antibody may have two antigen-binding sites through two pairs of VH/VL domains, both binding the same target antigen.

The term “multispecific” with respect to an ABM or antibody as described herein shall refer to a molecule having at least two binding sites specifically binding at least two different target antigens. The term shall include bispecific antibodies or molecules with 2 or more specificities to bind more than one target antigen, e.g. through at least 2, 3, 4 or even more binding sites.

For example, a bispecific antibody may bind one target antigen through one pair of VH/VL domains (Fv region), and another target antigen by a second pair of VH/VL domains (Fv region). A bispecific antibody typically is composed of four different antibody chains, i.e. two HCs and two LCs, such that two different CDR binding sites are formed by heterodimerization (pairing) of a first HC with a first LC and a second HC with a second LC.

The term “antigen” or “target” as used herein shall in particular include all antigens and target molecules capable of being recognised by a binding site of an antibody (also referred to as paratope). Specifically preferred antigens as targeted by the binding molecule as described herein are those antigens, which have already been proven to be or are capable of being immunologically or therapeutically relevant, especially those, for which a clinical efficacy has been tested. The term “target” or “antigen” as used herein shall in particular comprise molecules selected from the group consisting of (human or other animal) tumor associated receptors and soluble tumor associated antigens, which are self antigens, such as receptors located on the surface of tumor cells or cytokines or growth factors that are abundantly present in the circulation of cancer patients and associated with such tumor. Further antigens may be of pathogen origin, e.g. microbial or viral pathogens.

The target antigen is either recognized as a whole target molecule or as a fragment of such molecule, especially substructures, e.g. a polypeptide or carbohydrate structure of targets, generally referred to as “epitopes”, e.g. B-cell epitopes, T-cell epitope), which are immunologically relevant, i.e., are also recognisable by natural or monoclonal antibodies. The term “epitope” as used herein shall in particular refer to a molecular structure which may completely make up a specific binding partner or be part of a specific binding partner to a binding site of an ABM or antibody as described herein. The term epitope may also refer to haptens. Chemically, an epitope may either be composed of a carbohydrate, a peptide, a fatty acid, an organic, biochemical or inorganic substance or derivatives thereof and any combinations thereof. If an epitope is a polypeptide, it will usually include at least 3 amino acids, preferably 8 to 50 amino acids, and more preferably between about 10-20 amino acids in the peptide. There is no critical upper limit to the length of the peptide, which could comprise nearly the full length of a polypeptide sequence of a protein. Epitopes can be either linear or conformational epitopes. A linear epitope is comprised of a single segment of a primary sequence of a polypeptide or carbohydrate chain. Linear epitopes can be contiguous or overlapping. Conformational epitopes are comprised of amino acids or carbohydrates brought together by folding of the polypeptide to form a tertiary structure and the amino acids are not necessarily adjacent to one another in the linear sequence. Specifically, epitopes are at least part of diagnostically relevant molecules, i.e. the absence or presence of an epitope in a sample is qualitatively or quantitatively correlated to either a disease or to the health status of a patient or to a process status in manufacturing or to environmental and food status. Epitopes may also be at least part of therapeutically relevant molecules, i.e. molecules which can be targeted by the specific binding domain which changes the course of the disease.

Specific embodiments refer to naturally-occurring antigens or epitopes, or synthetic (artificial) antigens of epitopes. Artificial antigens which are derivatives of naturally-occurring antigens may have the advantage of an increased antigenicity or stability, which is relevant for being recognized as a binding partner for the specific ABM or antibody.

As used herein, the term “specificity” or “specific binding” refers to a binding reaction which is determinative of the cognate ligand of interest in a heterogeneous population of molecules. Thus, under designated conditions (e.g. immunoassay conditions), the ABM or antibody described herein binds to its particular target and does not bind in a significant amount to other molecules present in a sample. The specific binding means that binding is selective in terms of target identity, high, medium or low binding affinity or avidity, as selected. Selective binding is usually achieved if the binding constant or binding dynamics is at least 10 fold different, preferably the difference is at least 100 fold, and more preferred a least 1000 fold.

The term “variable binding region” also called “CDR region” as used herein refers to molecules with varying structures capable of binding interactions with antigens. Those molecules can be used as such or integrated within a larger protein, thus forming a specific region of such protein with binding function. The varying structures can be derived from natural repertoires of binding proteins such as from immunoglobulins or antibodies. The varying structures can as well be produced by randomisation techniques, in particular those described herein. These include mutagenized CDR or non-CDR regions (e.g. structural loop regions of constant antibody domains), loop regions of antibody variable domains or constant domains, in particular CDR loops of antibodies. Typically, binding structures of the ABM or antibody described herein are formed by such variable binding regions.

The term “cytotoxic” or “cytotoxic activity” as used for the purpose of an ABM or antibody described herein shall refer to any specific molecule directed against cellular antigens that, when bound to the antigen, activates programmed cell death and triggers apoptosis. Specific antibodies are effective by its activity on effector cells resulting in activation of cytotoxic T-cells or cells which mediate antibody-dependent cell cytotoxicity (ADCC), complement dependent cytotoxicity (CDC) and/or cellular phagocytosis (ADCP). Specific antibodies kill antibody-coated target cells by apoptosis inducing programmed cell death and/or by binding to Fc receptors of effector cells mediating ADCC and/or CDC activity.

An ABM or antibody described herein may or may not exhibit Fc effector function. Fc may recruit complement and aid elimination of a target antigen or a target cell through binding a surface antigen by formation of immune complexes.

Specific antibodies may be devoid of an active Fc moiety or Fc effector function, thus, either composed of antibody domains that do not contain an Fc part of an antibody or that do not contain an Fcgamma receptor binding site, or comprising antibody domains lacking Fc effector function, e.g. by modifications to reduce Fc effector functions, in particular to abrogate or reduce ADCC and/or CDC activity. Alternative antibodies may be engineered to incorporate modifications to increase Fc effector functions, in particular to enhance ADCC and/or CDC activity.

Such modifications may be effected by mutagenesis, e.g. mutations in the Fcgamma receptor binding site or by derivatives or agents to interfere with ADCC and/or CDC activity of an antibody format, so to achieve reduction or increase of Fc effector function.

The term “antigen-binding site” or “binding site” refers to the part of an ABM or antibody that participates in antigen binding. The antigen binding site of an antibody is typically formed by amino acid residues of the N-terminal variable (“V”) regions of the heavy (“H”) and/or light (“L”) chains, or the variable domains thereof. Three highly divergent stretches within the V regions of the heavy and light chains, referred to as “hypervariable regions”, are interposed between more conserved flanking stretches known as framework regions. The antigen-binding site provides for a surface that is complementary to the three-dimensional surface of a bound epitope or antigen, and the hypervariable regions are referred to as “complementarity-determining regions”, or “CDRs.” The binding site incorporated in the CDRs is herein also called “CDR binding site”.

The term “expression” is understood in the following way. Nucleic acid molecules containing a desired coding sequence of an expression product such as e.g. an ABM or antibody as described herein, and control sequences such as e.g. a promoter in operable linkage, may be used for expression purposes. Hosts transformed or transfected with these sequences are capable of producing the encoded proteins. In order to effect transformation, the expression system may be included in a vector; however, the relevant DNA may also be integrated into the host chromosome. Specifically the term refers to a host cell and compatible vector under suitable conditions, e.g. for the expression of a protein coded for by foreign DNA carried by the vector and introduced to the host cell.

Coding DNA is a DNA sequence that encodes a particular amino acid sequence for a particular polypeptide or protein such as e.g. an antibody. Promoter DNA is a DNA sequence which initiates, regulates, or otherwise mediates or controls the expression of the coding DNA. Promoter DNA and coding DNA may be from the same gene or from different genes, and may be from the same or different organisms. Recombinant cloning vectors will often include one or more replication systems for cloning or expression, one or more markers for selection in the host, e.g. antibiotic resistance, and one or more expression cassettes.

“Vectors” used herein are defined as DNA sequences that are required for the transcription of cloned recombinant nucleotide sequences, i.e. of recombinant genes and the translation of their mRNA in a suitable host organism.

An “expression cassette” refers to a DNA coding sequence or segment of DNA that code for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct”.

Expression vectors comprise the expression cassette and additionally usually comprise an origin for autonomous replication in the host cells or a genome integration site, one or more selectable markers (e.g. an amino acid synthesis gene or a gene conferring resistance to antibiotics such as zeocin, kanamycin, G418 or hygromycin), a number of restriction enzyme cleavage sites, a suitable promoter sequence and a transcription terminator, which components are operably linked together. The term “vector” as used herein includes autonomously replicating nucleotide sequences as well as genome integrating nucleotide sequences. A common type of vector is a “plasmid”, which generally is a self-contained molecule of double-stranded DNA that can readily accept additional (foreign) DNA and which can readily be introduced into a suitable host cell. A plasmid vector often contains coding DNA and promoter DNA and has one or more restriction sites suitable for inserting foreign DNA. Specifically, the term “vector” or “plasmid” refers to a vehicle by which a DNA or RNA sequence (e.g. a foreign gene) can be introduced into a host cell, so as to transform the host and promote expression (e.g. transcription and translation) of the introduced sequence.

The term “host cell” as used herein shall refer to primary subject cells transformed to produce a particular recombinant protein, such as an ABM or antibody as described herein, and any progeny thereof. It should be understood that not all progeny are exactly identical to the parental cell (due to deliberate or inadvertent mutations or differences in environment), however, such altered progeny are included in these terms, so long as the progeny retain the same functionality as that of the originally transformed cell. The term “host cell line” refers to a cell line of host cells as used for expressing a recombinant gene to produce recombinant polypeptides such as recombinant antibodies. The term “cell line” as used herein refers to an established clone of a particular cell type that has acquired the ability to proliferate over a prolonged period of time. Such host cell or host cell line may be maintained in cell culture and/or cultivated to produce a recombinant polypeptide.

The term “isolated” or “isolation” as used herein with respect to a nucleic acid, an antibody or other compound shall refer to such compound that has been sufficiently separated from the environment with which it would naturally be associated, so as to exist in “substantially pure” form. “Isolated” does not necessarily mean the exclusion of artificial or synthetic mixtures with other compounds or materials, or the presence of impurities that do not interfere with the fundamental activity, and that may be present, for example, due to incomplete purification. In particular, isolated nucleic acid molecules encoding the ABM or antibody described herein are also meant to include codon-optimized variants of naturally occurring nucleic acid sequences to improve expression in a certain host cell, or those chemically synthesized.

With reference to nucleic acids, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An “isolated nucleic acid” (either DNA or RNA) may further represent a molecule produced directly by biological or synthetic means and separated from other components present during its production.

With reference to polypeptides or proteins, such as isolated antibodies, the term “isolated” shall specifically refer to compounds that are free or substantially free of material with which they are naturally associated such as other compounds with which they are found in their natural environment, or the environment in which they are prepared (e g. cell culture) when such preparation is by recombinant DNA technology practiced in vitro or in vivo. Isolated compounds can be formulated with diluents or adjuvants and still for practical purposes be isolated—for example, the polypeptides or polynucleotides can be mixed with pharmaceutically acceptable carriers or excipients when used in diagnosis or therapy.

The term “recombinant” as used herein shall mean “being prepared by or the result of genetic engineering”. Alternatively, the term “engineered” is used. For example, an antibody or antibody domain may be engineered to produce a variant by engineering the respective parent sequence to produce a modified antibody or domain. A recombinant host specifically comprises an expression vector or cloning vector, or it has been genetically engineered to contain a recombinant nucleic acid sequence, in particular employing nucleotide sequence foreign to the host. A recombinant protein is produced by expressing a respective recombinant nucleic acid in a host. The term “recombinant antibody”, as used herein, includes antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant antibodies comprise antibodies engineered to include rearrangements and mutations which occur, for example, during antibody maturation.

Once antibodies with the desired structure are identified, such antibodies can be produced by methods well-known in the art, including, for example, hybridoma techniques or recombinant DNA technology.

In the hybridoma method, a mouse or other appropriate host animal, such as a hamster, is immunised to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell.

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

Recombinant monoclonal antibodies can, for example, be produced by isolating the DNA encoding the required antibody chains and transfecting a recombinant host cell with the coding sequences for expression, using well-known recombinant expression vectors, e.g. the plasmids or expression cassette(s) comprising the nucleotide sequences encoding the ABM or antibody described herein. Recombinant host cells can be prokaryotic and eukaryotic cells, such as those described above.

According to a specific aspect, the nucleotide sequence may be used for genetic manipulation to humanise the antibody or to improve the affinity, or other characteristics of the antibody. For example, the constant region may be engineered to more nearly resemble human constant regions to avoid immune response, if the antibody is used in clinical trials and treatments in humans. It may be desirable to genetically manipulate the antibody sequence to obtain greater affinity to the target antigen. It will be apparent to one of skill in the art that one or more polynucleotide changes can be made to the antibody and still maintain its binding ability to the target antigen.

The production of antibody molecules, by various means, is generally well understood. U.S. Pat. No. 6,331,415 (Cabilly et al.), for example, describes a method for the recombinant production of antibodies where the heavy and light chains are expressed simultaneously from a single vector or from two separate vectors in a single cell. Wibbenmeyer et al., (1999, Biochim Biophys Acta 1430(2):191-202) and Lee and Kwak (2003, J. Biotechnology 101:189-198) describe the production of monoclonal antibodies from separately produced heavy and light chains, using plasmids expressed in separate cultures of E. coli. Various other techniques relevant to the production of antibodies are provided in, e.g., Harlow, et al., ANTIBODIES: A LABORATORY MANUAL, Cold Spring Harbor Laboratory Press, Cold Spring Harbor, N.Y., (1988).

Monoclonal antibodies are produced using any method that produces antibody molecules by continuous cell lines in culture. Examples of suitable methods for pre-paring monoclonal antibodies include the hybridoma methods of Kohler et al. (1975, Nature 256:495-497) and the human B-cell hybridoma method (Kozbor, 1984, J. Immunol. 133:3001; and Brodeur et al., 1987, Monoclonal Antibody Production Techniques and Applications, (Marcel Dekker, Inc., New York), pp. 51-63).

The ABM or antibody as described herein may be used for administration to treat a subject in need thereof.

The term “subject” as used herein shall refer to a warm-blooded mammalian, particularly a human being or a non-human animal. Thus, the term “subject” may also particularly refer to animals including dogs, cats, rabbits, horses, cattle, pigs and poultry. In particular the ABM or antibody described herein is provided for medical use to treat a subject or patient in need of prophylaxis or treatment of a disease condition. The term “patient” includes human and other mammalian subjects that receive either prophylactic or therapeutic treatment. The term “treatment” is thus meant to include both prophylactic and therapeutic treatment.

Specifically, the ABM or antibody described herein is provided in substantially pure form. The term “substantially pure” or “purified” as used herein shall refer to a preparation comprising at least 50% (w/w), preferably at least 60%, 70%, 80%, 90% or 95% of a compound, such as a nucleic acid molecule or an antibody. Purity is measured by methods appropriate for the compound (e.g. chromatographic methods, polyacrylamide gel electrophoresis, HPLC analysis, and the like).

The term “therapeutically effective amount”, used herein interchangeably with any of the terms “effective amount” or “sufficient amount” of a compound, e.g. an ABM or antibody described herein, is a quantity or activity sufficient to, when administered to the subject effect beneficial or desired results, including clinical results, and, as such, an effective amount or synonym thereof depends upon the context in which it is being applied.

An effective amount is intended to mean that amount of a compound that is sufficient to treat, prevent or inhibit such diseases or disorder. In the context of disease, therapeutically effective amounts of the ABM or antibody as described herein are specifically used to treat, modulate, attenuate, reverse, or affect a disease or condition that benefits from the interaction of the antibody with its target antigen.

The amount of the compound that will correspond to such an effective amount will vary depending on various factors, such as the given drug or compound, the pharmaceutical formulation, the route of administration, the type of disease or disorder, the identity of the subject or host being treated, and the like, but can nevertheless be routinely determined by one skilled in the art.

The ABM or antibody described herein may specifically be used in a pharmaceutical composition. Therefore, a pharmaceutical composition is provided which comprise an ABM or antibody as described herein and a pharmaceutically acceptable carrier or excipient, e.g. an artificial carrier or excipient which does not naturally occur together with an immunoglobulin in a body fluid, or which naturally occurs together with an immunoglobulin, yet is provided in a preparation containing the carrier or excipient in a different amount or ratio.

Pharmaceutical compositions described herein can be administered as a bolus injection or infusion or by continuous infusion. Pharmaceutical carriers suitable for facilitating such means of administration are well-known in the art.

Pharmaceutically acceptable carriers generally include any and all suitable solid or liquid substances, solvents, dispersion media, coatings, isotonic and absorption delaying agents, and the like that are physiologically compatible with an ABM or antibody described herein. Further examples of pharmaceutically acceptable carriers include sterile water, saline, phosphate buffered saline, dextrose, glycerol, ethanol, and the like, as well as combinations of any thereof.

In one such aspect, an ABM or antibody can be combined with one or more carriers appropriate a desired route of administration. Antibodies may be, e.g. admixed with any of lactose, sucrose, starch, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, polyvinyl alcohol, and optionally further tableted or encapsulated for conventional administration. Alternatively, an ABM or antibody may be dissolved in saline, water, polyethylene glycol, propylene glycol, carboxymethyl cellulose colloidal solutions, ethanol, corn oil, peanut oil, cotton-seed oil, sesame oil, tragacanth gum, and/or various buffers. Other carriers, adjuvants, and modes of administration are well known in the pharmaceutical arts. A carrier may include a controlled release material or time delay material, such as glyceryl monostearate or glyceryl distearate alone or with a wax, or other materials well known in the art.

Additional pharmaceutically acceptable carriers are known in the art and described in, e.g. REMINGTON'S PHARMACEUTICAL SCIENCES. Liquid formulations can be solutions, emulsions or suspensions and can include excipients such as suspending agents, solubilizers, surfactants, preservatives, and chelating agents.

Pharmaceutical compositions are contemplated wherein an ABM or antibody described herein and one or more therapeutically active agents are formulated. Stable formulations are prepared for storage by mixing said ABM or antibody having the desired degree of purity with optional pharmaceutically acceptable carriers, excipients or stabilizers, in the form of lyophilized formulations or aqueous solutions. The formulations to be used for in vivo administration are specifically sterile, preferably in the form of a sterile aqueous solution. This is readily accomplished by filtration through sterile filtration membranes or other methods. The ABM or antibody and other therapeutically active agents disclosed herein may also be formulated as immunoliposomes, and/or entrapped in microcapsules.

Administration of the pharmaceutical composition comprising an ABM or antibody described herein, may be done in a variety of ways, including orally, subcutaneously, intravenously, intranasally, intraotically, transdermally, mucosal, topically, e.g., gels, salves, lotions, creams, etc., intraperitoneally, intramuscularly, intrapulmonary, vaginally, parenterally, rectally, or intraocularly.

Examplary formulations as used for parenteral administration include those suitable for subcutaneous, intramuscular or intravenous injection as, for example, a sterile solution, emulsion or suspension.

The invention specifically provides for exemplary ABM and antibodies as detailed in the examples provided herein. Further antibody variants are feasible, e.g. including functional variants of the exemplified antibodies, e.g. where the Fc is further engineered to improve the structure and function of the molecule, or where antibodies comprising different CDR binding sites or with different specificity are produced, in particular, wherein two different Fv regions are obtained.

The foregoing description will be more fully understood with reference to the following examples. Such examples are, however, merely representative of methods of practicing one or more embodiments of the present invention and should not be read as limiting the scope of invention.

### Examples

### Example 1: B10v5×hu225M SEED

A bispecific antibody with IgG structure is described. The B10v5-Fab binds to human c-MET while the hu225M-Fab binds to human EGFR (epidermal growth factor receptor). The interface between the hu225M light chain and the hu225M heavy chain harbours mutations that direct both light chains of the bispecific IgG to their cognate heavy chains. The CH3 domains of the antibody are replaced by SEED domains (either called SEED-AG or SEED-GA, Davis et al. 2010 and US 20070287170 A1) to enforce heterodimerisation of the heavy chains. LC-ESI-MS analysis is used to confirm the correct assembly of all four chains. In the following, the term BxM will be used to describe this bispecific IgG.

All following chains were cloned separately into the vector pTT5 (National Research Council Canada) for expression in a mammalian system.

The hu225M heavy chain with SEED-GA was termed hu225M_HC_GA (SEQ ID 6):

(SEQ ID 7)
MKLPVRLLVLMFWIPASLSEVQLVQSGAEVKKPGASVKVSCKASGFSL
TNYGVHWMRQAPGQGLEWIGVIWSGGNTDYNTPFTSRVTITSDKSTST
AYMELSSLRSEDTAVYYCARALTYYDYEFAYWGQGTLVTVSSASTKGP
SVFPLAPSSKSTSGGTAALGCLVKDYFPEPVTVSWNSGALTSGVHTFP
AVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLN
GKEYKCKVSNKALPAPIEKTISKAKGQPREPQVYTLPPPSEELALNEL
VTLTCLVKGFYPSDIAVEWLQGSQELPREKYLTWAPVLDSDGSFFLYS
ILRVAAEDWKKGDTFSCSVMHEALHNHYTQKSLDRSPGK
underlined: signal peptide MKLPVRLLVLMFWIPASLS

The hu225M light chain was termed hu225M_LC (SEQ ID 8):

(SEQ ID 7)
MKLPVRLLVLMFWIPASLSDIQMTQSPSSLSASVGDRVTITCRASQSI
GTNIHWYQQKPGKAPKLLIKYASESISGVPSRFSGSGYGTDFTLTISS
LQPEDVATYYCQQNYNWPTTFGQGTKVEIKRTVAAPSVFIFPPSDEQL
KSGTASVVCLLNNFYPREAKVQWKVDNALQSGNSQESVTEQDSKDSTY
SLSSTLTLSKADYEKHKVYACEVTHQGLSSPVTKSFNRGEC
underlined: signal peptide MKLPVRLLVLMFWIPASLS

The B10v5 heavy chain with SEED-AG was termed B10v5_HC_AG (SEQ ID 9):

(SEQ ID 10)
METDTLLLWVLLLWVPGSTGEVQLVQSGGGLVQPGGSLRLSCAASGFT
FSSYAMSWVRQAPGKGLEWVSAISGSGGSTYYADSVKGRFTISRDNSK
NTLYLQMNSLRAEDTAVYYCAKDRRITHTYWGQGTLVTVSSASTKGPS
VFPLAPSSKSTSGGTAALGCLVKDYFPEPVTVSWNSGALTSGVHTFPA
VLQSSGLYSLSSVVTVPSSSLGTQTYICNVNHKPSNTKVDKRVEPKSC
DKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNG
KEYKCKVSNKALPAPIEKTISKAKGQPFRPEVHLLPPSREEMTKNQVS
LTCLARGFYPKDIAVEWESNGQPENNYKTTPSRQEPSQGTTTFAVTSK
LTVDKSRWQQGNVFSCSVMHEALHNHYTQKTISLSPGK
underlined: signal peptide METDTLLLWVLLLWVPGSTG

The B10v5 light chain was termed B10v5_LC (SEQ ID 11):

(SEQ ID 10)
METDTLLLWVLLLWVPGSTGEPVLTQPPSVSVAPGETATIPCGGDSLG
SKIVHWYQQRPGQAPLLVVYDDAARPSGIPERFSGSKSGTTATLTISS
VEAGDEADYFCQVYDYHSDVEVFGGGTKLTVLGQPKAAPSVTLFPPSS
EELQANKATLVCLISDFYPGAVTVAWKADSSPVKAGVETTTPSKQSNN
KYAASSYLSLTPEQWKSHKSYSCQVTHEGSTVEKTVAPTECS
underlined: signal peptide METDTLLLWVLLLWVPGSTG

Introduction of Interface Mutations into hu225M

The mutations were introduced by site-directed mutagenesis using QuikChange Lightning Site-Directed Mutagenesis Kit (#210519, Agilent Technologies) according to the manufacturer's protocol. The mutation K26D was introduced into CH1 of hu225M_HC_AG and the mutation T18R was introduced into CL of hu225M_LC. Successful introduction of the mutation was confirmed by sequencing the gene of interest.

The hu225M heavy chain with mutation K26D was termed hu225M_HC_resQ28_GA (SEQ ID 12):

(SEQ ID 7)
MKLPVRLLVLMFWIPASLSEVQLVQSGAEVKKPGASVKVSCKASGFSL
TNYGVHWMRQAPGQGLEWIGVIWSGGNTDYNTPFTSRVTITSDKSTST
AYMELSSLRSEDTAVYYCARALTYYDYEFAYWGQGTLVTVSSASTKGP
SVFPLAPSSKSTSGGTAALGCLVDDYFPEPVTVSWNSGALTSGVHTFP
AVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLN
GKEYKCKVSNKALPAPIEKTISKAKGQPREPQVYTLPPPSEELALNEL
VTLTCLVKGFYPSDIAVEWLQGSQELPREKYLTWAPVLDSDGSFFLYS
ILRVAAEDWKKGDTFSCSVMHEALHNHYTQKSLDRSPGK
underlined: signal peptide MKLPVRLLVLMFWIPASLS

The hu225M light chain with mutation T18R was termed hu225M_LC_MB40 (SEQ ID 13)

(SEQ ID 7)
MKLPVRLLVLMFWIPASLSDIQMTQSPSSLSASVGDRVTITCRASQSI
GTNIHWYQQKPGKAPKLLIKYASESISGVPSRFSGSGYGTDFTLTISS
LQPEDVATYYCQQNYNWPTTFGQGTKVEIKRTVAAPSVFIFPPSDEQL
KSGRASVVCLLNNFYPREAKVQWKVDNALQSGNSQESVTEQDSKDSTY
SLSSTLTLSKADYEKHKVYACEVTHQGLSSPVTKSFNRGEC
underlined: signal peptide MKLPVRLLVLMFWIPASLS

Expression and Purification of BxM Wildtype and Mutant BxM MaB40 in Expi293FTM Cells

BxM was expressed using Expi293FTM cells and ExpiFectamine™ 293 Transfection kit (thermoFisher, A14525) according to the manufacturer's protocol. Two different transfections were set up:

| Name of antibody | chains |
|---|---|
| BxM wt | B10v5_HC_AG + |
| | B10v5_LC + |
| | hu225M_HC_GA + |
| | hu225M_LC |
| BxM MaB40 | B10v5_HC_AG + |
| | B10v5_LC + |
| | hu225M_HC_resQ28_GA + |
| | hu225M_LC_MB40 |

The DNA encoding each chain was on four different plasmids. The molar ratio of the plasmids during transfection was 2:1:1:1 (B10v5 heavy chain:B10v5 light chain:hu225M heavy chain:hu225M light chain).

The mutant BxM MaB40 contained the mutation K26D on CH1 of hu225M (hu225M_HC_resQ28_GA, SEQ ID 12) and T18R on CL of hu225M (hu225M_LC_MB40, SEQ ID 13) while BxM wildtype did not contain any mutation. The cultures were spun down and the supernatants containing the protein of interest were filtered through a 0.22 μm filter and purified using Montage antibody purification kit and Spin columns with PROSEP-A Media (Merck-Millipore, LSK2ABA20) according to the manufacturer's instructions. The purified BxM wildtype and BxM MaB40 were concentrated using Amicon ultra-15, 10 kDa MWCO, and then dialysed using Slide-A-Lyser Dialysis Cassettes 0.5-3 ml 7,000 MWCO (ThermoFisher, #66370) against PBS. In total, both BxM wildtype and BxM MaB40 were expressed, purified and analysed twice independently. Both replicates led to similar results.

LC-ESI-MS Analysis to Analyse Chain Pairing

The N-glycans of both samples were released using PNGase F prior to the measurement with an LC-ESI-MS system. The masses of all ten possible chain pairing variants were calculated and the mass spectra were analysed for their presence. A mispaired variant that contains four different chains but with both light chains binding to their non-cognate heavy chains (i.e. BxM with swapped light chains) has the same mass as the correctly assembled BxM and is, therefore, not distinguishable from the correctly paired BxM. However, if mispairing of one or of both light chains to their non-cognate heavy chains is undetectable then one can statistically exclude the presence of BxM with swapped light chains. Similarly, if mispairing of both light chains is detected only in low amounts (<5% relative abundance) then said mispaired variant will be present only in negligible amounts (<1%).

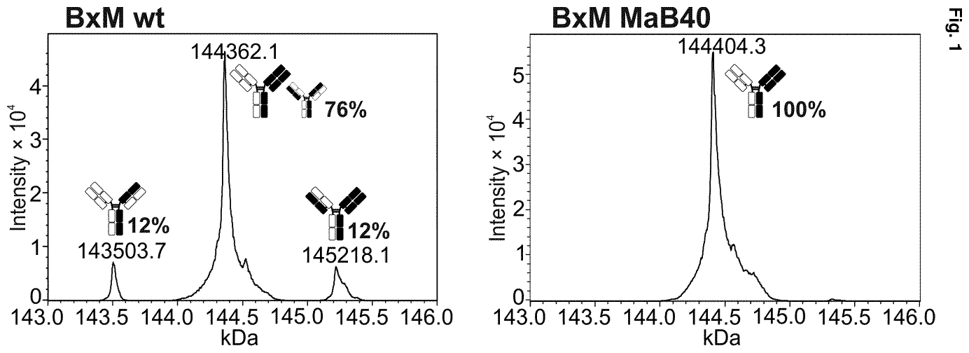

No heavy chain homodimers were detected in any of the samples (FIG. 1). In BxM wildtype, both light chains were able to bind to their non-cognate heavy chain in similar amounts (12% relative abundance in both cases) resulting in only 76% of correctly paired BxM (not counting in BxM with swapped light chains). In BxM MaB40 mispairing was undetectable and only the correctly paired bispecific IgG was detected.

In conclusion, the introduction of interface mutations led to the complete disappearance of mispairing of light to heavy chains.

BxM wt

| | theoretical | detected | maximum | relative |
|---|---|---|---|---|
| | mass | mass | peak | abundance in |
| pairing variant | (Da) | (Da) | intensity | % |
| B10v5_HC_AG + | 144363.5 | 144362.1 | 45416 | 76 |
| B10v5_LC + | | | | |
| hu225M_HC_GA + | | | | |
| hu225M_LC | | | | |
| B10v5_HC_AG + | 143507.5 | 143503.7 | 7124 | 12 |
| 2x B10v5_LC + | | | | |
| hu225M_HC_GA | | | | |
| B10v5_HC_AG + | 145219.5 | 145218.1 | 7161 | 12 |
| hu225M_HC_GA + | | | | |
| 2x hu225M_LC | | | | |

BxM MaB40

| | theoretical | detected | maximum | relative |
|---|---|---|---|---|
| | mass | mass | peak | abundance in |
| pairing variant | (Da) | (Da) | intensity | % |
| B10v5_HC_AG + | 144405.5 | 144404.3 | 54902 | 100 |
| B10v5_LC + | | | | |
| hu225M_HC_resQ28_GA + | | | | |
| hu225M_LC_MB40 | | | | |
| B10v5_HC_AG + | 143494.4 | not detected | 0 | 0 |
| 2x B10v5_LC + | | | | |
| hu225M_HC_resQ28_GA | | | | |
| B10v5_HC_AG + | 145316.5 | not detected | 0 | 0 |
| hu225M_HC_resQ28_GA + | | | | |
| 2x hu225M_LC_MB40 | | | | |

Size Exclusion Chromatography HPLC (HPLC-SEC)

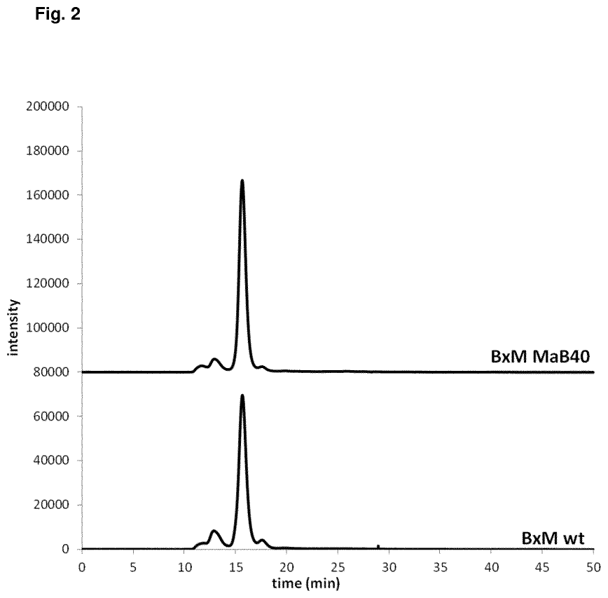

BxM wildtype and BxM MaB40 were analysed using SEC. Both chromatograms showed a main peak at 15.6 min which was expected for an IgG. Signs of aggregation were detectable in the mutant as well as the wildtype (FIG. 2).

Thermal Shift Assay to Determine Thermal Stability

A thermal shift assay was performed using the real time PCR system Step One Plus. The concentration of BxM wildtype and BxM MaB40 was 1 μM in PBS and the dye Sypro Orange (Invitrogen) at 20× final concentration was used. Both samples were measured in triplicates. The thermogram of BxM wildtype revealed two unfolding events at 64.8° C. and 74.5° C. The thermogram of BxM MaB40 revealed two unfolding events at 64.6° C. and 74.6° C. Thus, the interface mutations do not compromise the thermal stability of the protein.

Affinity of BxM to its Antigens

The affinity of BxM wildtype and MaB40 was analysed using an Octet system with biosensors coated with protein A. As antigens, the extracellular domains of cMET and EGFR were used. Three different concentrations of antibody were tested to determine the affinity. The KD of BxM wt to cMET was 0.35 nM and to EGFR 5.3 nM. The KD of BxM MaB40 to cMET was 0.42 nM and to EGFR 2.9 nM confirming that the interface mutations do not compromise the affinity of the antibody.

Simultaneous Binding of Both Antigens

The ability of BxM to bind to both of its antigens simultaneously was confirmed using an Octet System with streptavidin coated biosensors. First, the biosensors were submersed into a solution containing biotinylated cMET. After quenching and buffer change the biosensors were submersed in solutions either containing BxM wildtype or BxM MaB40. In both cases binding of the antibody to its first antigen was detected. Thereafter, the biosensors were submersed into a solution containing EGFR and binding to the second antigen was detected for wildtype and MaB40.

Impact of Interface Mutations on Yield in HEK293-6E

BxM wildtype and BxM MaB40 were expressed in HEK293-6E cells (National Research Council Canada) using transient transfection with polyethylenimine (PEI) according to standard techniques. The expressed IgGs were purified by protein A affinity chromatography and dialysed against PBS. The absorbance of both protein samples was measured at 280 nm to determine the concentration. In total, both proteins were expressed, purified and measured three times independently and the mean yield was calculated. The yield of BxM wildtype was 57.3 mg/L (±13.7 standard deviation) and the yield of BxM MaB40 was 58.9 mg/L (±7.3 standard deviation) demonstrating that the interface engineering has no detrimental effect on protein yield.

### Example 2: B10v5×OKT3 SEED

A bispecific antibody with IgG structure similar as described in Example 1 is described. The B10v5-Fab binds to human c-MET while the OKT3-Fab binds to human CD3. The interface between the OKT3 light chain and the OKT3 heavy chain harbours the same mutations as described in Example 1. In addition, mutations in the B10v5-Fab were introduced to further enforce the correct pairing of light to heavy chains. As above, SEED technology was applied for heterodimerisation of the heavy chains. LC-ESI-MS analysis was used to confirm the correct assembly of all four chains. In the following, the term BxO will be used to describe this bispecific IgG.

Cloning of Constructs

B10v5 heavy chain (SEQ ID 9) and light chain (SEQ ID 11) are described in Example 1.

All following chains were cloned separately into the vector pTT5 (National Research Council Canada) for expression in a mammalian system.

The OKT3 heavy chain was termed OKT3_HC_GA (SEQ ID 14)

(SEQ ID 7)
MKLPVRLLVLMFWIPASLSQVQLVQSGGGVVQPGRSLRLSCKASGYTF
TRYTMHWVRQAPGKGLEWIGYINPSRGYTNYNQKVKDRFTISRDNSKN
TAFLQMDSLRPEDTGVYFCARYYDDHYCLDYWGQGTPVTVSSASTKGP
SVFPLAPSSKSTSGGTAALGCLVKDYFPEPVTVSWNSGALTSGVHTFP
AVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLN
GKEYKCKVSNKALPAPIEKTISKAKGQPREPQVYTLPPPSEELALNEL
VTLTCLVKGFYPSDIAVEWLQGSQELPREKYLTWAPVLDSDGSFFLYS
ILRVAAEDWKKGDTFSCSVMHEALHNHYTQKSLDRSPGK
underlined: signal peptide MKLPVRLLVLMFWIPASLS

The OKT3 light chain was termed OKT3_LC (SEQ ID 15)

(SEQ ID 7)
MKLPVRLLVLMFWIPASLSDIQMTQSPSSLSASVGDRVTITCSASSSV
SYMNWYQQTPGKAPKRWIYDTSKLASGVPSRFSGSGSGTDYTFTISSL
QPEDIATYYCQQWSSNPFTFGQGTKLQITRTVAAPSVFIFPPSDEQLK
SGTASVVCLLNNFYPREAKVQWKVDNALQSGNSQESVTEQDSKDSTYS
LSSTLTLSKADYEKHKVYACEVTHQGLSSPVTKSFNRGEC
underlined: signal peptide MKLPVRLLVLMFWIPASLS

Introduction of Interface Mutations into OKT3 and B10v5

The mutations were introduced by site-directed mutagenesis using QuikChange Lightning Site-Directed Mutagenesis Kit (#210519, Agilent Technologies) according to the manufacturer's protocol as described above. In OKT3 the mutations K26D in CH1 and T18R in CL were introduced. In B10v5 the mutations A20L in CH1 and either F7S, F7A or F7V in CL were introduced. Successful introduction of the mutations was confirmed by sequencing the gene of interest.

The OKT3 heavy chain with mutation K26D was termed OKT3_HC_resQ28_GA (SEQ ID 16)

(SEQ ID 7)
MKLPVRLLVLMFWIPASLSQVQLVQSGGGVVQPGRSLRLSCKASGYTF
TRYTMHWVRQAPGKGLEWIGYINPSRGYTNYNQKVKDRFTISRDNSKN
TAFLQMDSLRPEDTGVYFCARYYDDHYCLDYWGQGTPVTVSSASTKGP
SVFPLAPSSKSTSGGTAALGCLVDDYFPEPVTVSWNSGALTSGVHTFP
AVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLN
GKEYKCKVSNKALPAPIEKTISKAKGQPREPQVYTLPPPSEELALNEL
VTLTCLVKGFYPSDIAVEWLQGSQELPREKYLTWAPVLDSDGSFFLYS
ILRVAAEDWKKGDTFSCSVMHEALHNHYTQKSLDRSPGK
underlined: signal peptide MKLPVRLLVLMFWIPASLS

The OKT3 light chain with mutation T18R was termed OKT3_LC_MB40 (SEQ ID 17)

(SEQ ID 7)
MKLPVRLLVLMFWIPASLSDIQMTQSPSSLSASVGDRVTITCSASSSV
SYMNWYQQTPGKAPKRWIYDTSKLASGVPSRFSGSGSGTDYTFTISSL
QPEDIATYYCQQWSSNPFTFGQGTKLQITRTVAAPSVFIFPPSDEQLK
SGRASVVCLLNNFYPREAKVQWKVDNALQSGNSQESVTEQDSKDSTYS
LSSTLTLSKADYEKHKVYACEVTHQGLSSPVTKSFNRGEC
underlined: signal peptide MKLPVRLLVLMFWIPASLS

The B10v5 heavy chain with mutation A20L was termed B10v5_HC_resQ203_AG (SEQ ID 18)

(SEQ ID 10)
METDTLLLWVLLLWVPGSTGEVQLVQSGGGLVQPGGSLRLSCAASGFT
FSSYAMSWVRQAPGKGLEWVSAISGSGGSTYYADSVKGRFTISRDNSK
NTLYLQMNSLRAEDTAVYYCAKDRRITHTYWGQGTLVTVSSASTKGPS
VFPLAPSSKSTSGGTALLGCLVKDYFPEPVTVSWNSGALTSGVHTFPA
VLQSSGLYSLSSVVTVPSSSLGTQTYICNVNHKPSNTKVDKRVEPKSC
DKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNG
KEYKCKVSNKALPAPIEKTISKAKGQPFRPEVHLLPPSREEMTKNQVS
LTCLARGFYPKDIAVEWESNGQPENNYKTTPSRQEPSQGTTTFAVTSK
LTVDKSRWQQGNVFSCSVMHEALHNHYTQKTISLSPGK
underlined: signal peptide METDTLLLWVLLLWVPGSTG

The B10v5 light chain with mutation F7S was termed B10v5_LC_MB9 (SEQ ID 19)

(SEQ ID 10)
METDTLLLWVLLLWVPGSTGEPVLTQPPSVSVAPGETATIPCGGDSLG
SKIVHWYQQRPGQAPLLVVYDDAARPSGIPERFSGSKSGTTATLTISS
VEAGDEADYFCQVYDYHSDVEVFGGGTKLTVLGQPKAAPSVTLSPPSS
EELQANKATLVCLISDFYPGAVTVAWKADSSPVKAGVETTTPSKQSNN
KYAASSYLSLTPEQWKSHKSYSCQVTHEGSTVEKTVAPTECS
underlined: signal peptide METDTLLLWVLLLWVPGSTG

The B10v5 light chain with mutation F7A was termed B10v5_LC_MB21 (SEQ ID 20)

(SEQ ID 10)
METDTLLLWVLLLWVPGSTGEPVLTQPPSVSVAPGETATIPCGGDSLG
SKIVHWYQQRPGQAPLLVVYDDAARPSGIPERFSGSKSGTTATLTISS
VEAGDEADYFCQVYDYHSDVEVFGGGTKLTVLGQPKAAPSVTLAPPSS
EELQANKATLVCLISDFYPGAVTVAWKADSSPVKAGVETTTPSKQSNN
KYAASSYLSLTPEQWKSHKSYSCQVTHEGSTVEKTVAPTECS
underlined: signal peptide METDTLLLWVLLLWVPGSTG

The B10v5 light chain with mutation F7V was termed B10v5_LC_MB45 (SEQ ID 21)

(SEQ ID 10)
METDTLLLWVLLLWVPGSTGEPVLTQPPSVSVAPGETATIPCGGDSLG
SKIVHWYQQRPGQAPLLVVYDDAARPSGIPERFSGSKSGTTATLTISS
VEAGDEADYFCQVYDYHSDVEVFGGGTKLTVLGQPKAAPSVTLVPPSS
EELQANKATLVCLISDFYPGAVTVAWKADSSPVKAGVETTTPSKQSNN
KYAASSYLSLTPEQWKSHKSYSCQVTHEGSTVEKTVAPTECS
underlined: signal peptide METDTLLLWVLLLWVPGSTG

Expression and Purification of BxO Wildtype and Mutants BxO MaB40, BxO MaB5/40, BxO MaB 21/40 and BxO MaB45/40 in HEK293-6E Cells

The bispecific antibodies were expressed in HEK293-6E cells using transient transfection with polyethylenimine (PEI) according to standard techniques. Four different transfections were set up:

| Name of antibody | chains |
|---|---|
| BxO wt | B10v5_HC_AG + |
| | B10v5_LC + |
| | OKT3_HC_GA + |
| | OKT3_LC |
| BxO MaB40 | B10v5_HC_AG + |
| | B10v5_LC + |
| | OKT3_HC_resQ28_GA + |
| | OKT3_LC_MB40 |
| BxO MaB5/40 | B10v5_HC_resQ203_AG + |
| | B10v5_LC_MB9 + |
| | OKT3_HC_resQ28_GA + |
| | OKT3_LC_MB40 |
| BxO MaB21/40 | B10v5_HC_resQ203_AG + |
| | B10v5_LC_MB21 + |
| | OKT3_HC_resQ28_GA + |
| | OKT3_LC_MB40 |
| BxO MaB45/40 | B10v5_HC_resQ203_AG + |
| | B10v5_LC_MB45 + |
| | OKT3_HC_resQ28_GA + |
| | OKT3_LC_MB40 |

The DNA encoding each chain was on four different plasmids. The molar ratio of the plasmids during transfection was 2:1:1:1 (B10v5 heavy chain:B10v5 light chain:OKT3 heavy chain:OKT3 light chain). The cultures were harvested 5 days post transfection by centrifugation and the supernatants were purified via protein A affinity chromatography. All samples were dialysed against PBS.

LC-ESI-MS Analysis to Analyse Chain Pairing

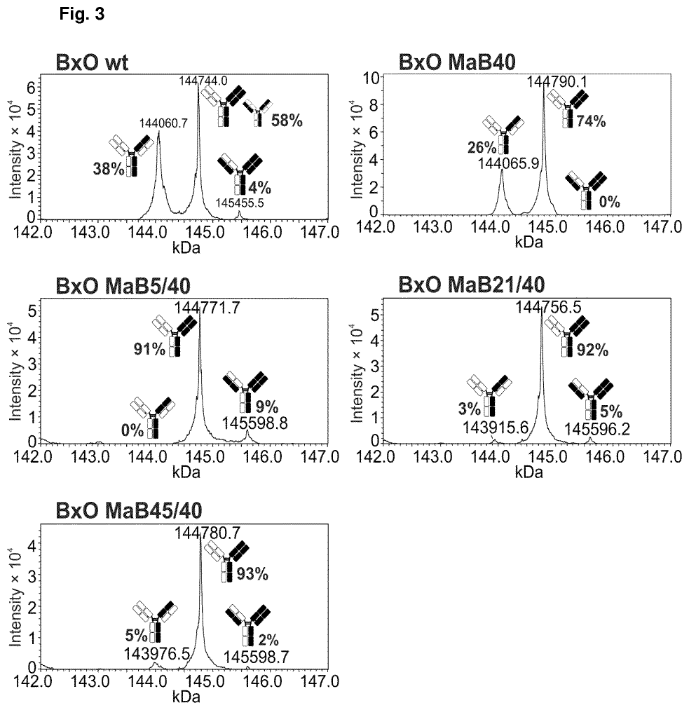

The N-glycans of both samples were released using PNGase F prior to the measurement. The analysis was performed as described in Example 1. The introduction of interface mutations led to a remarkable decrease in detectable mispairing of light to heavy chains (FIG. 3).

BxO wt

| | theoretical | detected | maximum | relative |
|---|---|---|---|---|
| | mass | mass | peak | abundance in |
| pairing variant | (Da) | (Da) | intensity | % |
| B10v5_HC_AG + | 144746.8 | 144744.0 | 61845 | 58 |
| B10v5_LC + | | | | |
| OKT3_HC_GA + | | | | |
| OKT3_LC | | | | |
| B10v5_HC_AG + | 144036.2 | 144060.7 | 40339 | 38 |
| 2x B10v5_LC + | | | | |
| OKT3_HC_GA | | | | |
| B10v5_HC_AG + | 145457.5 | 145455.5 | 4045 | 4 |
| OKT3_HC_GA + | | | | |
| 2x OKT3_LC | | | | |

BxO MaB40

| | theoretica | detected | maximum | relative |
|---|---|---|---|---|
| | mass | mass | peak | abundance in |
| pairing variant | (Da) | (Da) | intensity | % |
| B10v5_HC_AG + | 144788.8 | 144788.9 | 95970 | 74 |
| B10v5_LC + | | | | |
| OKT3_HC_resQ28_GA + | | | | |
| OKT3_LC_MB40 | | | | |
| B10v5_HC_AG + | 144023.1 | 144051.4 | 33344 | 26 |
| 2x B10v5_LC + | | | | |
| OKT3_HC_resQ28_GA | | | | |
| B10v5_HC_AG + | 145554.6 | not detectable | 0 | 0 |
| OKT3_HC_resQ28_GA + | | | | |
| 2x OKT3_LC_MB40 | | | | |

| | | | | |
|---|---|---|---|---|
| | theoretical | detected | maximum | relative |
| | mass | mass | peak | abundance in |
| pairing variant | (Da) | (Da) | intensity | % |

BxO MaB5/40

| | | | | |
|---|---|---|---|---|
| B10v5_HC_resQ203_AG + | 144770.8 | 144771.7 | 51479 | 91 |
| B10v5_LC_MB9 + | | | | |
| OKT3_HC_resQ28_GA + | | | | |
| OKT3_LC_MB40 | | | | |
| B10v5_HC_resQ203_AG + | 143945.0 | not detectable | 0 | 0 |
| 2x B10v5_LC_MB9 + | | | | |
| OKT3_HC_resQ28_GA | | | | |
| B10v5_HC_resQ203_AG + | 145596.7 | 145598.8 | 5272 | 9 |
| OKT3_HC_resQ28_GA + | | | | |
| 2x OKT3_LC_MB40 | | | | |

BxO MaB21/40

| | | | | |
|---|---|---|---|---|
| B10v5_HC_resQ203_AG + | 144754.8 | 144756.5 | 53125 | 92 |
| B10v5_LC_MB21 + | | | | |
| OKT3_HC_resQ28 GA + | | | | |
| OKT3_LC_MB40 | | | | |
| B10v5_HC_resQ203_AG + | 143913.0 | 143915.6 | 1695 | 3 |
| 2x B10v5_LC_MB21 + | | | | |
| OKT3_HC_resQ28_GA | | | | |
| B10v5_HC_resQ203_AG + | 145596.7 | 145596.2 | 2632 | 5 |
| OKT3_HC_resQ28_GA + | | | | |
| 2x OKT3_LC_MB40 | | | | |

BxO MaB45/40

| | theoretical | detected | maximum | relative |
|---|---|---|---|---|
| | mass | mass | peak | abundance in |
| pairing variant | (Da) | (Da) | intensity | % |
| B10v5_HC_resQ203_AG + | 144782.8 | 144780.7 | 43344 | 93 |
| B10v5_LC_MB45 + | | | | |
| OKT3_HC_resQ28_GA + | | | | |
| OKT3_LC_MB40 | | | | |
| B10v5_HC_resQ203_AG + | 143969.0 | 143976.5 | 2199 | 5 |
| 2x B10v5_LC_MB45 + | | | | |
| OKT3_HC_resQ28_GA | | | | |
| B10v5_HC_resQ203_AG + | 145596.7 | 145598.7 | 965 | 2 |
| OKT3_HC_resQ28_GA + | | | | |
| 2x OKT3_LC_MB40 | | | | |

Size Exclusion Chromatography HPLC (HPLC-SEC)

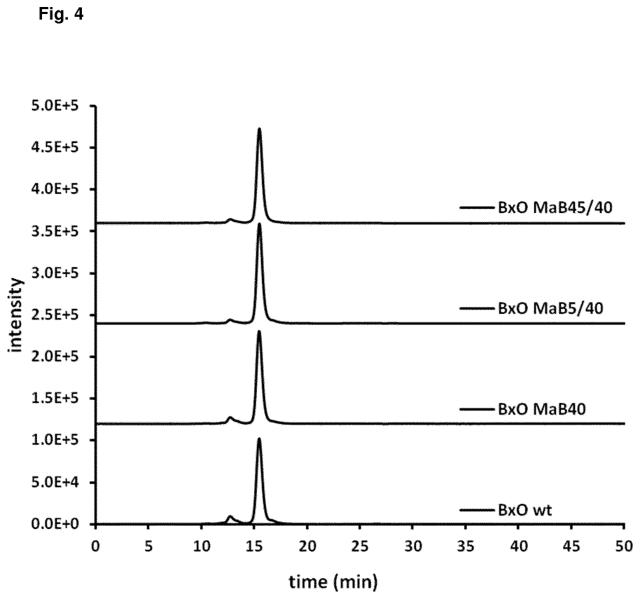

BxO wildtype, BxO MaB5/40 and BXO MaB45/40 were analysed using SEC. All chromatograms showed a main peak at 15.5 min which was expected for an IgG (FIG. 4). Signs of aggregation were detectable in the mutants as well as the wildtype in similar amounts.

Discussion

Example 1 describes the production of a bispecific antibody with IgG structure termed BxM, with or without mutations in the hu225M Fab. The analysis of BxM wildtype by LC-ESI-MS demonstrates the problem of producing a bispecific IgG without any engineering to enforce the correct pairing of the light chains to their cognate heavy chains. Both light chains were able to bind to their non-cognate heavy chain to a combined amount of 24% which in turn means only 76% of the purified protein sample was the correctly paired bispecific IgG.

To create the mutant BxM MaB40 only two point mutations, K26D in CH1 and T18R in CL, both in hu225M Fab, were introduced. These mutations were sufficient to inhibit the incorrect light-to-heavy chain pairing entirely. In contrast to previous reports (Lewis er al. 2014, Liu et al. 2015), no engineering of the variable domains was necessary. Therefore, the mutations of the present invention have the potential of being broadly applicable in various other bispecific antibodies. In addition, omitting any mutation in the variable domains limits the risk of affecting the affinity of the antibody to its antigen.

Further investigation revealed that the mutations introduced in BxM MaB40 had no detrimental effect on thermal stability as well as protein yield. Additionally, BxM MaB40 had an affinity to both its antigens similar to that of BxM wildtype and was able to bind to both antigens simultaneously as demonstrated by biolayer interferometry. Size exclusion chromatography revealed no differences between BxM MaB40 and BxM wildtype which proves that the mutations do not lead to increased aggregation or degradation of the antibody.

In order to assess if the identified mutations are of a generic use a different bispecific antibody, BxO, was constructed as shown in example 2. Similar to example 1, BxO without mutations (wildtype) was compared to BxO with the mutations K26D in CH1 and T18R in CL of the OKT3 Fab (termed BxO MaB40). Analysis of BxO wildtype by LC-ESI-MS revealed that mispairing of the light chains to their non-cognate heavy chains was more prevalent than mispairing detected in BxM wildtype. In total, more than 40% of detected IgGs exhibited mispairing in the Fab resulting in less than 60% correctly paired BxO. The analysis of BxO MaB40 showed that the introduction of the aforementioned mutations again had a considerable effect on the pairing behaviour of the light chains. 74% of detected IgGs in BxO MaB40 were correctly paired. To further enforce the correct assembly of chains, supportive mutations were introduced in the other Fab of BxO, i.e. the B10v5 Fab, leading to the creation of BxO MaB5/40, BxO MaB21/40 and BxO MaB45/40. In all three mutants containing supportive mutations the amount of correctly paired bispecific IgGs was vastly improved (>90% relative abundance in LC-ESI-MS).

The analysis of BxO wildtype, BxO MaB5/40 and BXO MaB45/40 by size exclusion chromatography demonstrated that the present mutations do not have a detrimental effect on the antibody regarding aggregation or degradation.

### Example 3: Surface Exposure of Amino Acid Side Chains in Positions at the Interface Between CH1 and CL

The GETAREA program (Fraczkiewicz et al. 1998, J. Comp. Chem., 19, 319-333) was used to calculate solvent accessible surface area or solvation energy of proteins. Atomic coordinates of the human IgG1 Fab fragment 1 DFB.pdb, which is a human monoclonal antibody Fab fragment against gp41 of human immunodeficiency virus type 1 with wildtype CH1 and CL domains (He et al. 1992, Proc.Natl.Acad.Sci.USA 89: 7154-7158), were supplied to the program as input. A probe radius of 1.4 Angstrom was applied. The output of the program for residues ALA20 and LYS26 in the CH1 domain and PHE7 and THR18 in the CL domain is shown in the Table I below.

The contributions from backbone and sidechain atoms are listed in the 4th and 5th columns respectively. The next column lists the ratio of side-chain surface area to the “random coil” value per residue. The “random coil” value of a residue X is the average solvent-accessible surface area of X in the tripeptide Gly-X-Gly in an ensemble of 30 random conformations. Residues are considered to be solvent exposed, if the ratio value exceeds 50%, buried if the ratio is less than 20%, and not buried, if the ratio is at least 20%. The “random coil” values for 20 amino acids are listed in Table II below.

TABLE I
GETAREA output for the surface exposure of amino acid
side chains in positions at the interface between
CH1 and CL. The numbering is according to IMGT

| Residue | Total | Apolar | Backbone | Sidechain | Ratio(%) | In/Out |
|---|---|---|---|---|---|---|
| CH1 | | | | | | |
| ALA 20 | 0.00 | 0.00 | 0.00 | 0.00 | 0.0 | i |
| LYS 26 | 7.42 | 7.42 | 0.00 | 7.42 | 4.5 | i |
| CL | | | | | | |
| PHE 7 | 2.30 | 2.20 | 0.17 | 2.13 | 1.2 | i |
| THR 18 | 40.63 | 27.06 | 0.00 | 40.63 | 38.3 | |

TABLE II
Random coil values of 20 amino acids

| | |
|---|---|
| ALA | 64.9 |
| ARG | 195.5 |
| ASN | 114.3 |
| ASP | 113.0 |
| CYS | 102.3 |
| GLN | 143.7 |
| GLU | 141.2 |
| HIS | 154.6 |
| ILE | 147.3 |
| GLY | 87.2 |
| LEU | 146.2 |
| LYS | 164.5 |
| MET | 158.3 |
| PHE | 180.1 |
| PRO | 105.2 |
| SER | 77.4 |
| THR | 106.2 |
| TRP | 224.6 |
| TYR | 193.1 |
| VAL | 122.3 |

From the results shown in Table I above, it can be seen that three of the four residues are buried (ratio (%) value of less than 5%), whereas residue THR18 in the CL domain has a ratio (%) value of 38.3 and is therefore not buried.

It was surprising to find out that mutating position THR18 in the CL domain, which is not buried within the CH/CH1 interface, leads to an improved pairing of the CL/CH1 domains. Improved pairing was particularly found when pairing a CL domain with a CH1 domain, wherein the amino acid residue at position 18 in the CL domain has opposite polarity to the amino acid resue at position 26 in the CH1 domain, as further described herein.

This was the more surprising because prior art engineering approaches applied certain criteria for selecting pairs of residues along the heavy and light chain interface to be replaced by charged residues with opposite polarity. According to such criteria according to the prior art (e.g., Liu et al. Journal Of Biological Chemistry 2015, 290:7535-7562; and WO2014/081955A1), it was deemed essential that all positions are buried.

Thus, position 18 in the CL domain is an exception to this prior art rule, and surprisingly contributes to the stability of the CL/CH1 domain pair despite of not being buried within the interface between CH1 and CL.

1. An antigen-binding molecule (ABM) comprising a cognate light chain/heavy chain (LC/HC) dimer of an antibody light chain (LC) composed of a VL and a CL antibody domain, associated to an antibody heavy chain (HC) comprising at least a VH and a CH1 antibody domain, which association is through pairing the VL and VH domains and the CL and CH1 domains, wherein the amino acids at the position 18 in the CL domain and at the position 26 in the CH1 domain are of opposite polarity, wherein numbering is according to the IMGT.

2. The ABM of claim 1, wherein
A
a) the CL domain is Ckappa comprising an amino acid sequence with at least 90% sequence identity to SEQ ID 1 which contains at least the point mutation T18X, wherein X is any of R, H, or K; and
b) the CH1 domain comprises an amino acid sequence with at least 90% sequence identity to SEQ ID 3 which contains at least the point mutation K26X, wherein X is any of D, or E;
or B
a) the CL domain is Clambda comprising an amino acid sequence with at least 90% sequence identity to SEQ ID 2 which contains at least the point mutation K18X, wherein X is any of D, or E; and
b) the CH1 domain comprises an amino acid sequence with at least 90% sequence identity to SEQ ID 3 wherein K at position 26 is not substituted by any other amino acid, or which contains at least the point mutation K26X, wherein X is any of R, or H;
or C
a) the CL domain is Clambda comprising an amino acid sequence with at least 90% sequence identity to SEQ ID 2 wherein K at position 18 is not substituted by any other amino acid, or which contains at least the point mutation K18X, wherein X is any of R, or H; and
b) the CH1 domain comprises an amino acid sequence with at least 90% sequence identity to SEQ ID 3 which contains at least the point mutation K26X, wherein X is any of D, or E;
wherein numbering is according to the IMGT.

3. The ABM of claim 1 or 2, which cognate LC/HC dimer comprises at least one interdomain disulfide bridge between the CL and CH1 domains.

4. The ABM of any of claims 1 to 3, which CL domain further comprises the point mutation F7X, wherein X is any of S, A, or V, and which CH1 domain further comprises the point mutation A20L, wherein numbering is according to the IMGT.

5. The ABM of any of claims 1 to 4, wherein the VL and VH domains do not contain any point mutation changing the polarity of an amino acid in the interface region.

6. The ABM of any of claims 1 to 5, wherein the HC further comprises at least one CH2 and at least one CH3 domain.

7. The ABM of any of claims 1 to 5, which is any of an antibody Fab or (Fab)₂ fragment, or a full-length antibody comprising an Fc part, preferably wherein the ABM is a full-length IgG antibody.

8. The ABM of any of claims 1 to 6, which is a heterodimeric antibody comprising a first and a second Fab arm recognizing different antigens or epitopes, wherein only one of the first and second Fab arms comprises the cognate LC/HC dimer.

9. The ABM of claim 8, wherein only one of the first and second Fab arms comprises
a) the point mutation F7X in the CL domain, wherein X is any of S, A, or V; and
b) the point mutation A20L in the CH1 domain;
wherein numbering is according to the IMGT.

10. The ABM of claim 8 or 9, wherein
A
a) said first Fab arm comprises the cognate LC/HC dimer which is characterized by the point mutations identified in any of claim 1 or 2, wherein the CL and CH1 domains further comprise the point mutations identified in claim 4; and
b) said second Fab arm does not comprise any of the point mutations of a), or
B
a) said first Fab arm comprises the cognate LC/HC dimer which is characterized by the point mutations identified in any of claim 1 or 2, wherein the CL and CH1 domains do not further comprise the point mutations identified in claim 4; and
b) said second Fab arm comprises the point mutations identified in claim 4.

11. The ABM of any of claims 8 to 10, which further comprises two HCs each comprising a CH2 and a CH3 domain which HCs dimerize into an Fc region, wherein the CH3 domains are engineered to introduce one or more of the following:
a) strand-exchange engineered domain (SEED) CH3 heterodimers that are composed of alternating segments of human IgA and IgG CH3 sequences;
b) one or more knob or hole mutations, preferably any of T366Y/Y407′T, F405A/T394′W, T366Y:F405A/T394W:Y407′T, T366W/Y407′A and S354C:T366W/Y349′C:T366′S:L368′A:Y407′V;
c) a cysteine residue in the first CH3 domain that is covalently linked to a cysteine residue in the second CH3 domain, thereby introducing an interdomain disulfide bridge, preferably linking the C-terminus of both CH3 domains;
d) one or more mutations where repulsive charge suppresses heterodimer formation, preferably any of: K409D/D399′K, K409D/D399′R, K409E/D399′K, K409E/D399′R, K409D:K392D/D399′K:E356′K or K409D:K392D:K370D/D399′K:E356′K:E357′K; and/or
e) one or more mutations selected for heterodimer formation and/or thermostability, preferably any of:
T350V:L351Y:F405A:Y407V/T350V:T366L:K392L:T394W,
T350V:L351Y:F405A:Y407V/T350V:T366L:K392M:T394W,
L351Y:F405A:Y407V/T366L:K392M:T394W,
F405A:Y407V/T366L:K392M:T394W, or
F405A:Y407V/T366L:T394W,
wherein numbering is according to the EU index of Kabat.

12. An isolated nucleic acid encoding the ABM of any of claims 1 to 11.

13. An expression cassette or vector incorporating the nucleic acid of claim 12.

14. A host cell comprising the nucleic acid of claim 12, or the expression cassette or vector of claim 13.

15. A method of producing the ABM of any of claims 1 to 11, by cultivating a host cell of claim 14 under conditions to express said ABM.


SEQUENCE LISTING (21 sequences)

SEQ ID NO: 1 - protein, 107 aa, Homo sapiens [sequence omitted]

SEQ ID NO: 2 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 3 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 4 - protein, 15 aa, Homo sapiens
EPKSCDKTHTCPPCP

SEQ ID NO: 5 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 471 aa, Artificial Sequence
  Antibody heavy chain
MKLPVRLLVLMFWIPASLSEVQLVQSGAEVKKPGASVKVSCKASGFSLTNYGVHWMRQAPGQGLEWIGVIWSGGNTDYNTPFTSRVTITSDKSTSTAYMELSSLRSEDTAVYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPPSEELALNELVTLTCLVKGFYPSDIAVEWLQGSQELPREKYLTWAPVLDSDGSFFLYSILRVAAEDWKKGDTFSCSVMHEALHNHYTQKSLDRSPGK

SEQ ID NO: 7 - protein, 19 aa, Artificial Sequence
  Signal peptide
MKLPVRLLVLMFWIPASLS

SEQ ID NO: 8 - protein, 233 aa, Artificial Sequence
  Antibody light chain
MKLPVRLLVLMFWIPASLSDIQMTQSPSSLSASVGDRVTITCRASQSIGTNIHWYQQKPGKAPKLLIKYASESISGVPSRFSGSGYGTDFTLTISSLQPEDVATYYCQQNYNWPTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 470 aa, Artificial Sequence
  Antibody heavy chain
METDTLLLWVLLLWVPGSTGEVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKDRRITHTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGK

SEQ ID NO: 10 - protein, 20 aa, Artificial Sequence
  Signal peptide
METDTLLLWVLLLWVPGSTG

SEQ ID NO: 11 - protein, 234 aa, Artificial Sequence
  Antibody light chain
METDTLLLWVLLLWVPGSTGEPVLTQPPSVSVAPGETATIPCGGDSLGSKIVHWYQQRPGQAPLLVVYDDAARPSGIPERFSGSKSGTTATLTISSVEAGDEADYFCQVYDYHSDVEVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 12 - protein, 471 aa, Artificial Sequence
  Antibody heavy chain
MKLPVRLLVLMFWIPASLSEVQLVQSGAEVKKPGASVKVSCKASGFSLTNYGVHWMRQAPGQGLEWIGVIWSGGNTDYNTPFTSRVTITSDKSTSTAYMELSSLRSEDTAVYYCARALTYYDYEFAYWGQGTLVTVSSASTKGPSVFPLAPSSKSTSGGTAALGCLVDDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPPSEELALNELVTLTCLVKGFYPSDIAVEWLQGSQELPREKYLTWAPVLDSDGSFFLYSILRVAAEDWKKGDTFSCSVMHEALHNHYTQKSLDRSPGK

SEQ ID NO: 13 - protein, 233 aa, Artificial Sequence
  Antibody light chain
MKLPVRLLVLMFWIPASLSDIQMTQSPSSLSASVGDRVTITCRASQSIGTNIHWYQQKPGKAPKLLIKYASESISGVPSRFSGSGYGTDFTLTISSLQPEDVATYYCQQNYNWPTTFGQGTKVEIKRTVAAPSVFIFPPSDEQLKSGRASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 471 aa, Artificial Sequence
  Antibody heavy chain
MKLPVRLLVLMFWIPASLSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPPSEELALNELVTLTCLVKGFYPSDIAVEWLQGSQELPREKYLTWAPVLDSDGSFFLYSILRVAAEDWKKGDTFSCSVMHEALHNHYTQKSLDRSPGK

SEQ ID NO: 15 - protein, 232 aa, Artificial Sequence
  Antibody light chain
MKLPVRLLVLMFWIPASLSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 471 aa, Artificial Sequence
  Antibody heavy chain
MKLPVRLLVLMFWIPASLSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASTKGPSVFPLAPSSKSTSGGTAALGCLVDDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPPSEELALNELVTLTCLVKGFYPSDIAVEWLQGSQELPREKYLTWAPVLDSDGSFFLYSILRVAAEDWKKGDTFSCSVMHEALHNHYTQKSLDRSPGK

SEQ ID NO: 17 - protein, 232 aa, Artificial Sequence
  Antibody light chain
MKLPVRLLVLMFWIPASLSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGRASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 470 aa, Artificial Sequence
  Antibody heavy chain
METDTLLLWVLLLWVPGSTGEVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKDRRITHTYWGQGTLVTVSSASTKGPSVFPLAPSSKSTSGGTALLGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGK

SEQ ID NO: 19 - protein, 234 aa, Artificial Sequence
  Antibody light chain
METDTLLLWVLLLWVPGSTGEPVLTQPPSVSVAPGETATIPCGGDSLGSKIVHWYQQRPGQAPLLVVYDDAARPSGIPERFSGSKSGTTATLTISSVEAGDEADYFCQVYDYHSDVEVFGGGTKLTVLGQPKAAPSVTLSPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 20 - protein, 234 aa, Artificial Sequence
  Antibody light chain
METDTLLLWVLLLWVPGSTGEPVLTQPPSVSVAPGETATIPCGGDSLGSKIVHWYQQRPGQAPLLVVYDDAARPSGIPERFSGSKSGTTATLTISSVEAGDEADYFCQVYDYHSDVEVFGGGTKLTVLGQPKAAPSVTLAPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 21 - protein, 234 aa, Artificial Sequence
  Antibody light chain
METDTLLLWVLLLWVPGSTGEPVLTQPPSVSVAPGETATIPCGGDSLGSKIVHWYQQRPGQAPLLVVYDDAARPSGIPERFSGSKSGTTATLTISSVEAGDEADYFCQVYDYHSDVEVFGGGTKLTVLGQPKAAPSVTLVPPSSEELQANKATLVCLISDFYPGAVTVAWKADSSPVKAGVETTTPSKQSNNKYAASSYLSLTPEQWKSHKSYSCQVTHEGSTVEKTVAPTECS